(19) United States
(12) Patent Application Publication
(10) Pub. No.: US 2024/0226144 A1
(43) Pub. Date: Jul. 11, 2024

(54) METHODS OF PROTECTING AN ORGAN PRIOR TO SURGERY IN PATIENTS SUSCEPTIBLE TO POST-OPERATIVE COMPLICATIONS INCLUDING PHOTOSENSITIVITY

(71) Applicant: Renibus Therapeutics, Inc., Southlake, TX (US)

(72) Inventors: Donald Jeffrey Keyser, Southlake, TX (US); Bhupinder Singh, Phoenix, AZ (US); Stacey Ruiz, Lantana, TX (US)

(21) Appl. No.: 18/407,130

(22) Filed: Jan. 8, 2024

Related U.S. Application Data
(60) Provisional application No. 63/478,975, filed on Jan. 8, 2023.
(60) Provisional application No. 63/499,974, filed on May 3, 2023.
(60) Provisional application No. 63/595,699, filed on Nov. 2, 2023.
(60) Provisional application No. 63/601,700, filed on Nov. 21, 2023.
(60) Provisional application No. 63/602,216, filed on Nov. 22, 2023.

(51) Int. Cl. A61K 33/26; A61K 33/24; A61P 41/00

(52) U.S. Cl. CPC A61K 33/26; A61K 33/24; A61P 41/00

(57) ABSTRACT

Methods of treating patients before a scheduled insult such as surgy by administering a combination of stannic protoporfin and iron sucrose are provided. The present methods provide protection to photosensitive patients and those susceptible to post-operative complications.

The present application claims the benefits of U.S. Provisional Application No. 63/478,975 filed Jan. 8, 2023; U.S. Provisional Application No. 63/449,974 filed May 3, 2023; U.S. Provisional Application No. 63/595,699 filed Nov. 2, 2023; U.S. Provisional Application No. 63/601,700 filed Nov. 21, 2023; and U.S. Provisional Application No. 63/602,216 filed Nov. 22, 2023, each of which is hereby incorporated by references in their entirety.

### BACKGROUND

Injury to a bodily organ can elicit protective responses by the organ such that it is able to better protect itself should injurious events (i.e., insults) continue or re-occur. For example, a bout of kidney injury can evoke protective responses that, after an 18-hour lag time, protect the kidney against subsequent, more severe forms of kidney damage. This protection can last for an extended period of time (days to weeks). This protective phenomenon is known in the art as “ischemic preconditioning” or “acquired cytoresistance.”

One thought has been to use the phenomenon of acquired cytoresistance to preemptively protect organs, especially when a known insult is imminent. Various pharmacological preconditioning strategies such as volatile anesthetics, noble gases, dexmedetomidine, and levosimendan, have been evaluated in randomized controlled trials in cardiac surgery with largely neutral results. The present inventors previously found that acquired cytoresistance could be induced to protect organs before an insult, such as exposure to surgery, cardiopulmonary bypass, or radiocontrast toxicity administrations. The present inventors have previously disclosed that administration of a combination of an iron compound and a protoporphyrin, specifically stannic protoporfin (e.g., stannsoporfin or SnPP), may be utilized to protect an organ from an insult, such as surgery. See U.S. Pat. No. 9,844,563 filed Dec. 19, 2017, entitled “COMPOSITIONS, KITS, AND METHODS TO INDUCE ACQUIRED CYTORESISTANCE USING STRESS PROTEIN INDUCERS.”

Photosensitivity issues upon administration of SnPP are well documented. See Land et al., “Photophysical studies of tin(IV)-protoporphyrin: Potential phototoxicity of a chemotherapeutic agent proposed for the prevention of neonatal jaundice,” Proc. Natl. Acad. Sci. USA, Vol. 85, pp. 5249-5253 (1988). Photosensitivity appears to have been a factor that previously led others to abandon SnPP for tin mesoporphyrin (SnMP) in the treatment of neonatal jaundice. Furthermore, the present inventors cited photosensitivity as an exclusion criteria in the original study design of the Phase II clinical trial, NCT04564833, entitled “Effect of RBT-1 on Preconditioning Response Biomarkers in Subjects Undergoing CABG and/or Cardiac Valve Surgery.” Specifically, this study excluded patients with “History of photosensitivity or active skin disease that, in the opinion of the Investigator, could be worsened by RBT-1.”

There remains a need to develop methods for protecting an organ prior to surgery in photosensitive patients, and to improve post-operative outcomes in patients undergoing a surgical operation.

### SUMMARY OF THE INVENTION

The present invention in one aspect relates to a method of reducing post operative complications of a human patient from injury based on a scheduled or anticipated surgical operation, the method comprising administering to the human patient a composition comprising a therapeutically effective amount of (i) an iron compound; and (ii) stannic protoporfin in a dose of 20-80 mg before the surgical operation, wherein the human patient is susceptible to photodermatoses.

In aspects where the patient is photosensitive, the method may involve applying sunscreen to the patient's skin within six days after the scheduled or anticipated surgical operation. The sunscreen may be applied to the patient's skin before any expected sun exposure within six days after the scheduled or anticipated surgical operation. In one aspect, the human patient may be instructed to or may desire to reduce or eliminate sun exposure between the administration of the therapeutically effective composition and the scheduled or anticipated surgical operation. The human patient may apply sunscreen when sunlight when exposure is expected for at least 6 days after the scheduled or anticipated surgical operation. In one aspect, sunscreen is administered when sun exposure is expected after the therapeutically effective composition is administered. The sunscreen may have a sun protection factor (SPF) of 50+.

In another aspect, the present invention relates to a method of reducing post operative complications of a human patient from injury based on a scheduled or anticipated insult to an organ of the human patient, the method comprising: (a) administering to the patient a therapeutically effective composition comprising (i) an iron compound; and (ii) stannic protoporfin, and (b) applying sunscreen to the human patient's skin around the time of administering to the human patient the therapeutically effective composition or within six days after the scheduled or anticipated surgical operation. The sunscreen may be applied to the patient's skin before any expected sun exposure within six days after the scheduled or anticipated surgical operation. In one aspect, the human patient may be instructed to or may desire to reduce or eliminate sun exposure between the administration of the therapeutically effective composition and the scheduled or anticipated surgical operation. The human patient may apply sunscreen when sunlight when exposure is expected for at least 6 days after the scheduled or anticipated surgical operation. In one aspect, sunscreen is administered when sun exposure is expected after the therapeutically effective composition is administered. The sunscreen may have a sun protection factor (SPF) of 50+.

In another aspect, the present invention relates to a method of reducing hospital readmission for cardiopulmonary purposes of a human patient after a surgical operation by at least 60% comprising administering to the patient a therapeutically effective composition comprising an amount of (i) an iron compound; and (ii) stannic protoporfin before the surgical operation. The method may result in a rate of readmission for cardiopulmonary purposes that is reduced by at least 70%, or more specifically 72%. The human patient may be selected from a patient that has an elevated risk for hospital readmission for cardiopulmonary purposes after receiving and CABG and/or heart valve surgery.

In another aspect, the present invention relates to a method of reducing post operative complications of a human patient from injury based on a scheduled or anticipated surgical operation comprising administering to the patient a therapeutically effective composition comprising an amount of (i) an iron compound; and (ii) stannic protoporfin before the surgical operation, wherein the post operative complications include: (a) greater than three days in the intensive care unit, (b) greater than 24 hours on a ventilator, (c) readmission for cardiopulmonary surgery, (d) need for a blood transfusion, (e) new onset post-operative atrial fibrillation (POAF) during hospitalization, or a combination of two or more of (a)-(e). The human patient may be selected from a patient that has an elevated risk post-operative complications including one or more of the above-listed complications after receiving and CABG and/or heart valve surgery.

In any of the above embodiments, the stannic protoporfin may be administered in a dose of 20-80 mg, more preferably at a dose of 30-70 mg, or a dose of 45 mg. The iron sucrose may be administered at a dose of 190-290 mg, or preferably at a dose of 240 mg. In one embodiment, the stannic protoporfin is administered at a dose of 45 mg and iron compound is iron sucrose is administered at a dose of 240 mg.

In any of the above embodiments, the iron compound may be present in an aqueous pharmaceutical composition comprising: iron sucrose; bicarbonate; and a pharmaceutically acceptable aqueous carrier. The stannic protoporfin may be present in an aqueous pharmaceutical composition comprising: a total level of impurities of 1.5% or less, as measured by gas chromatography. In one aspect, the preparation of the therapeutically effective composition for administration may comprise combining in an intravenous bag: (a) an aqueous iron pharmaceutical composition comprising: iron sucrose; bicarbonate; and a pharmaceutically acceptable aqueous carrier; and (b) a stannic protoporfin composition having a total level of impurities of 1.5% or less, as measured by gas chromatography.

In one aspect, the therapeutically effective amount of stannic protoporfin may be administered at a dose of 0.5-0.8 mg/kg, more preferably 0.6-0.7 mg/kg, and particularly at 0.63 mg/kg. In another aspect, the therapeutically effective amount of iron sucrose may be 2-6 times the amount by weight of stannic protoporfin, or more preferably 3-4 times the amount of stannic protoporfin.

In one aspect, the scheduled or anticipated surgical operation is surgery to an organ. The organ may be heart, kidney, liver or lung. In one aspect, the iron compound and the stannic protoporfin are administered at least 24 hours before the scheduled or anticipated surgical operation to the organ occurs. In one aspect, it is desirable that the iron compound and the stannic protoporfin be administered no more than 48 hrs before the scheduled or anticipated surgical operation to the organ occurs.

In one aspect, the scheduled or anticipated surgical operation may be a surgery comprising coronary artery bypass graft (CABG), cardiac valve, or combined CABG/valve surgery on cardiopulmonary bypass (CPB). In another aspect, the scheduled or anticipated surgical operation is organ transplant surgery. In the case of organ transplantation, the method may reduce or eliminate the need for a blood product to be administered after the scheduled or anticipated surgical operation. This may include whole blood products from a donor.

### BRIEF DESCRIPTION OF THE DRAWINGS

FIG. 1 shows a flowchart illustrating the design of the study assessing RBT-1 to reduce post-operative complications in patients undergoing cardiothoracic surgery;

FIG. 2 shows a statistically significant increase in cytoprotective response biomarkers HO-1, Ferritin, and IL-10 with RBT-1;

FIG. 3 on the left graph shows the percentage of subjects in each group that were on a ventilator for more than 24 hours post cardiac surgery and on the right graph shows the percentage of subjects that where in the ICU for greater than 3 days;

FIG. 4 shows the average time spent for each group on a ventilator, in the ICU, and in the hospital following cardiac surgery;

FIG. 5 shows the improvement in the mean composition of hospitalization in patients treated with RBT-1;

FIG. 6 shows the median Troponin I levels through the first day post-operation; and

FIG. 7 shows the improvement of postoperative outcomes in patients treated with RBT-1 undergoing cardiac surgery;

FIG. 8 shows the improvement of postoperative outcomes in patients treated with RBT-1 undergoing cardiac surgery;

FIG. 9 shows a flow diagram of patient populations according to Example 2.

FIG. 10 shows the mean composite of the maximum pre-operative change from baseline in HO-1, IL-10, and ferritin from Example 2.

FIG. 11 shows assessment of hospitalization course by time on ventilator and length of stay (LOS) in the ICU and hospital according to Example 2.

FIG. 12 shows the primary outcome—preconditioning response in accordance with Example 3.

FIG. 13 shows the ventilator, ICU and hospitalization course in accordance with Example 3.

FIG. 14 shows post-operative troponin I in accordance with Example 3.

FIG. 15 is a table showing the primary outcome for interim population in accordance with Example 3.

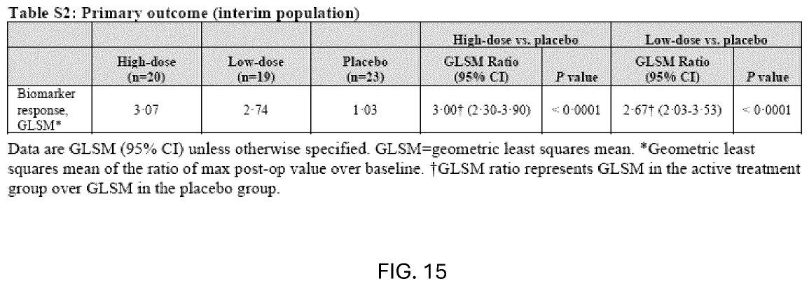

FIG. 16 is a table showing baseline characteristics for the safety population in accordance with Example 3.

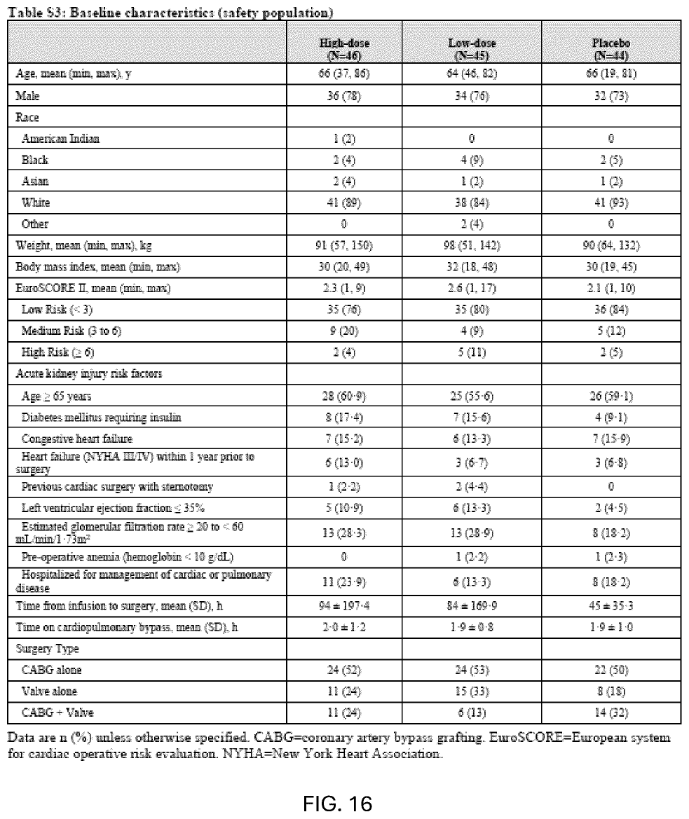

FIG. 17 is a table showing baseline characteristics for the modified intent-to-treat population in accordance with Example 3.

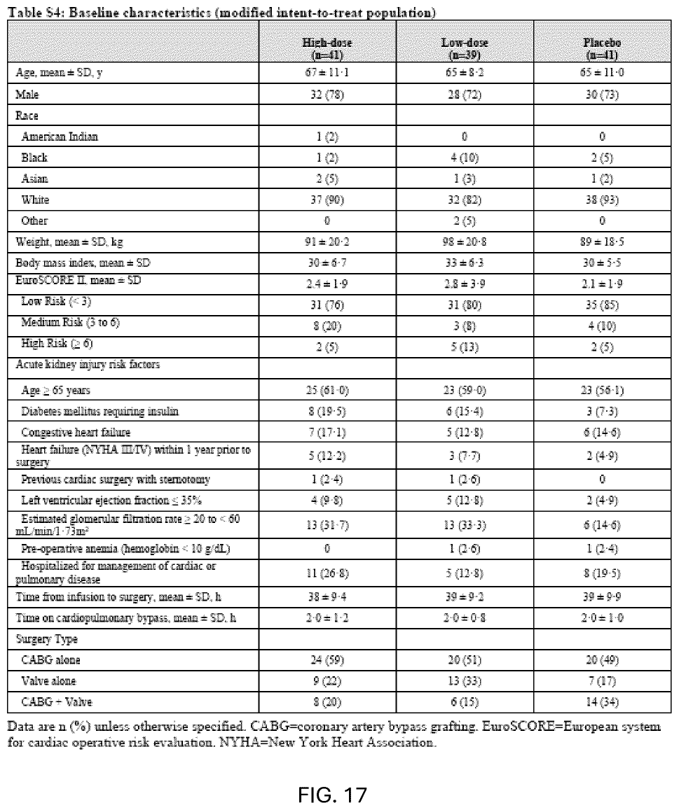

FIG. 18 is a table showing other prespecified outcomes for the modified intent-to-treat population in accordance with Example 3.

FIG. 19 is a table showing hierarchical composite outcome (win ratio) in accordance with Example 3.

FIG. 20 shows a comparison of ventilator, ICU times, and cardiopulmonary readmissions for Phase 2 patients undergoing CABG and/or valve surgery on cardiopulmonary bypass.

FIG. 21 shows the design for a Phase III study of RBT-1.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

For convenience, certain terms employed in the specification, examples and claims are collected here. Unless defined otherwise, all technical and scientific terms used in this disclosure have the same meanings as commonly understood by one of ordinary skill in the art to which this disclosure belongs.

“RBT-1” is synonymous with comprises a combination of stannic protoporfin and iron sucrose that has been used to generate data disclosed in this application.

The iron sucrose used in RBT-1 is an iron sucrose composition that can be readily combined with tin protoporphyrin (SnPP) to form a stable solution that can be administered to a patient, as described in U.S. Pat. No. 11,292,813 filed Apr. 5, 2022, entitled “NOVEL IRON COMPOSITIONS AND METHODS OF MAKING AND USING THE SAME,” the subject matter of which is incorporated herein by reference. The composition, also known as RBT-3, is an aqueous iron pharmaceutical composition comprising: iron sucrose; bicarbonate; and a pharmaceutically acceptable aqueous carrier, wherein the iron sucrose is present in pharmaceutically effective amount for providing a protective effect to a patient's organs the iron being present in both iron (II) and iron (III) form, the pharmaceutical composition has a pH greater than 9, a concentration of iron (II) of 0.05% w/v to 0.41% w/v, and the iron sucrose has a MW according to GPC of between 33,000 and 38,000 Daltons. RBT-3 (iron sucrose) is a low molecular weight iron nanoparticle that has the potential to rapidly restore iron levels and improve blood product utilization in cardiac surgery and/or ER patients. RBT-3 has also demonstrated the potential to mitigate cisplatin induced nephrotoxicity in preclinical models. We are currently exploring opportunities to further the clinical development of RBT-3 in these potential indications.

The iron sucrose composition can be prepared, for example, by dissolving enough iron sucrose complex in water (ca 3.5 L) to give a 12 mg/mL (expressed as iron) solution when diluted to 6.0 L. The amount of iron sucrose needed was calculated for the final volume of liquid, 6100 mL (6.1 L) so that the final concentration is 12 mg/mL. This requires 73.2 g of iron. The use potency of iron sucrose is 0.0550. Thus, 73.2 g/0.0550 or 1331 g±1 g of iron sucrose is needed. Iron sucrose, 1331 g±1 g, was weighed directly into a 6.0 L Erlenmeyer flask. Approximately 3-3.5 L of water is added to the Erlenmeyer flask, and the contents of the flask are stirred.

Sodium bicarbonate is added in an amount such that the final sodium bicarbonate concentration is 10 mg/mL when diluted to 6.0 L. Sodium bicarbonate, 109.8±0.1 g, is weighed and added to the 6.0 L flask. Sodium chloride is added in an amount such that the final sodium chloride concentration is 9.0 mg/mL upon dilution. Sodium chloride, 54.9±0.1 g, is weighed and added to the 6.0 L flask. The suspension is stirred for 30-120 minutes to give a black opaque solution. The pH of the solution is monitored with a pH meter while 1 M sodium hydroxide is added in small portions until pH 10.30 is reached and remains stable. Sodium hydroxide, 40.0+0.1 g, was added to a 1.0 L Erlenmeyer flask. 1.0±0.1 L of water is added to the 1.0 L Erlenmeyer flask and stirred until all of the sodium hydroxide dissolved. A pH probe is affixed to monitor the pH of the 6.0 L Erlenmeyer flask and the sodium hydroxide is added in <100 mL portions until the pH=10.3±0.1. The solution is then stirred for 10 minutes. The pH is checked again after 10 minutes and if necessary adjusted to within pH=10.3±0.1.

The solution is then transferred to a volumetrically accurate flask and diluted to 6.1 L with water. A 2 L volumetric flask is used twice to transfer exactly 4 L of the 10.3 pH solution to a 6 L Erlenmeyer flask. The remaining 10.3 pH solution is diluted to 2 L in a volumetric flask and added to the 6 L Erlenmeyer flask. The 100 mL graduated cylinder is used to add 100±0.1 mL to the 6.0 L Erlenmeyer, and the resulting solution is stirred for 10 minutes.

The resulting product solution appears dark red to brown. Two isotopes of iron are present in the sample preparation in a ratio consistent with that of the standard preparation. The resulting material had a pH of 10.3, which is within the preferred limits of 10.1-10.4. The resultant material had 11.5/11.6 parts per thousand (mg/mL) iron according to SOP 174472, which determines iron through inductively coupled plasma-mass spectroscopy.

The stannic protoporfin used in RBT-1 is a high purity composition as described in U.S. Provisional Application No. 63/394,146, filed Aug. 1, 2022, entitled “NOVEL SN-PROTOPORPHYRIN COMPOSITIONS, METHODS OF MAKING, AND USES THEREOF,” the subject matter of which is incorporated herein by reference. The Sn-protoporphyrin composition comprises a compound of Formula (I)

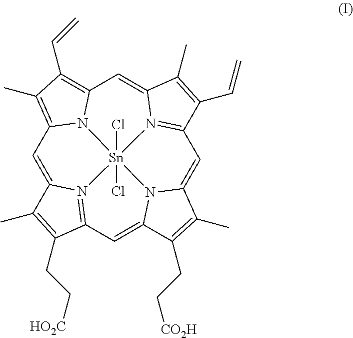

wherein the composition comprises a total level of impurities of 1.5% or less, as measured by gas chromatography. The Sn-protoporphyrin (IX) is made using a two-step process as follows:

Hemin was dissolved in hot formic acid, then iron powder was added in aliquots over 20 min. The resulting mixture was heated and stirred for 30 min, then filtered through Celite. The filtrate was added to a stirring aqueous solution of NH₄OAc to precipitate the desired product, which was filtered and dried. This crude material was dissolved in hot pyridine and the hot solution was filtered through Celite. The purified product precipitated from the filtrate upon cooing and was recovered by filtration.

Stannous chloride was dissolved in pyridine under inert atmosphere, glacial acetic acid was added, and the mixture is heated at 50° C. Protoporphyrin IX was then added and stirred and heated for a minimum of 24 hours and monitored for completion by HPLC. The reaction was cooled to room temperature and filtered. The product was then triturated first with water, then 2 M HCl(aq) and then again with water. An IPC was conducted to determine if a pyridine/AcOH recrystallization, followed by an additional water trituration. The product was then dried to remove residual solvents.

The present inventors surprising discovered through human trials that clinical outcomes can be improved by reducing the amount of stannic protoporfin used. The efficacy exhibited a U-shaped curve and lowering the amount of SnPP was unexpectedly found to increase efficacy. Specifically, the analysis of data in the human clinical trials disclosed herein demonstrate that the amount of stannic protoporfin thought to be effective and desirable of 90 mg was less efficacious compared to a lower dose of 45 mg. In addition, the high purity SnPP used may have also had some role in increasing the effectiveness of the composition. Moreover, the present inventors found that complications related to photosensitivity could be avoided and/or reduced by using a lower dose of SnPP without lowering the effectiveness for preventing post-operative complications related to a surgical procedure. This led the inventors to develop methods for treating patients who were susceptible to photosensitivity with RBT-1.

In one aspect, the present inventors discovered that a lower dose of SnPP could be utilized and that doing so unexpectedly allowed the inclusion of photosensitive patients. The previous literature suggested that photosensitive patients should not be administered SnPP. The present inventors found that the claim methods could use a lower dose of SnPP with photosensitive patients while maintaining the efficacy of RBT-1 for protecting an organ from scheduled insult, e.g., CABG surgery. The present inventors found that the use of sunscreen by a photosensitive patient along with the lower dose of 45 mg SnPP could enable participation in the study.

In another aspect, the present inventors found that patents who were at risk from rehospitalization after CABG surgery could benefit from the lower-dose administration of SnPP. The rate of rehospitalization was decreased by at least 60% relative to placebo, and in cases the rate of rehospitalization was reduced by at least 70%, or more specifically 72%. Other post-operative complications were found to be improved by way of the lower dose administration of SnPP. Those complications include: (a) greater than three days in the intensive care unit, (b) greater than 24 hours on a ventilator, (c) readmission for cardiopulmonary surgery, (d) need for a blood transfusion, (c) new onset post-operative atrial fibrillation (POAF) during hospitalization, or a combination of two or more of (a)-(c). The human patient may be selected from a patient that has an elevated risk post-operative complications including one or more of the above-listed complications after receiving and CABG and/or heart valve surgery.

In certain embodiments, the stannic protoporfin may be administered in a dose of 20-80 mg, more preferably at a dose of 30-70 mg, or a dose of 45 mg. The iron sucrose may be administered at a dose of 190-290 mg, or preferably at a dose of 240 mg. In one embodiment, the stannic protoporfin is administered at a dose of 45 mg and iron compound is iron sucrose is administered at a dose of 240 mg.

In certain embodiments, the iron compound may be present in an aqueous pharmaceutical composition comprising: iron sucrose; bicarbonate; and a pharmaceutically acceptable aqueous carrier. The stannic protoporfin may be present in an aqueous pharmaceutical composition comprising: a total level of impurities of 1.5% or less, as measured by gas chromatography. In one aspect, the preparation of the therapeutically effective composition for administration may comprise combining in an intravenous bag: (a) an aqueous iron pharmaceutical composition comprising: iron sucrose; bicarbonate; and a pharmaceutically acceptable aqueous carrier; and (b) a stannic protoporfin composition having a total level of impurities of 1.5% or less, as measured by gas chromatography.

In certain embodiments, the therapeutically effective amount of stannic protoporfin may be administered at a dose of 0.5-0.8 mg/kg, more preferably 0.6-0.7 mg/kg, and particularly at 0.63 mg/kg. In another aspect, the therapeutically effective amount of iron sucrose may be 2-6 times the amount by weight of stannic protoporfin, or more preferably 3-4 times the amount of stannic protoporfin.

In one aspect, the scheduled or anticipated surgical operation is surgery to an organ. The organ may be heart, kidney, liver or lung. In one aspect, the iron compound and the stannic protoporfin are administered at least 24 hours before the scheduled or anticipated surgical operation to the organ occurs. In one aspect, it is desirable that the iron compound and the stannic protoporfin be administered no more than 48 hrs before the scheduled or anticipated surgical operation to the organ occurs.

In one aspect, the scheduled or anticipated surgical operation may be a surgery comprising coronary artery bypass graft (CABG), cardiac valve, or combined CABG/valve surgery on cardiopulmonary bypass (CPB). In another aspect, the scheduled or anticipated surgical operation is organ transplant surgery. In the case of organ transplantation, the method may reduce or eliminate the need for a blood product to be administered after the scheduled or anticipated surgical operation. This may include whole blood products from a donor.

### Example 1

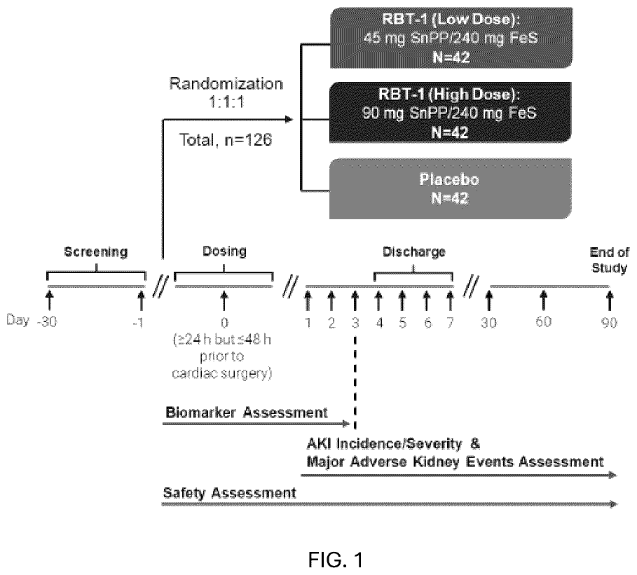

FIG. 1 shows the study design of administering RBT-1 (FeS+SnPP) at a low dose of 45 mg stannic protoporfin (SnPP)/240 mg iron sucrose (FeS) to 42 subjects and at a high dose 90 mg SnPP/240 mg FeS to 42 subjects while observing an addition 42 subjects with a placebo for a total of 126 subjects. The study was a multicenter, double-blind, placebo controlled, Phase 2 study in subjects undergoing coronary artery bypass graft (CABG) and/or cardiac valve surgery on cardiopulmonary bypass. The subjects were administered the doses prior to undergoing the cardiac surgery. The primary objective of the study is to determine the effect of RBT-1 in generating a preconditioning response, measured by a compositive of plasma biomarkers through Day 1 pre-surgery. These biomarkers include heme oxygenase-1 (HO-1), ferritin, and interleukin-10 (IL-10). Key secondary and exploratory objectives of the study include the effect of RBT-1 on the recovery of subjects by measuring days on a ventilator, days in intensive care unit (ICU), hospital length of stay, incidence of acute kidney events (AKI), incidence of major adverse kidney events (MAKE), hospital readmission rate, and safety.

The subjects came from 19 sites across the U.S., Canada, and Australia. The overall study population was not enriched for events. The subjects were randomized at a site level to account for differences in standard of care. The safety population consisted of all subjects who received the study drug. The ITT population consisted of subjects who received the study drug and had biomarker assessments performed per protocol for the primary assessment. The MITT population consisted of ITT subjects who underwent surgery within protocol defined timeframe (all clinical outcomes endpoints are based on this group).

The total number of subjects enrolled in this study was 152 subjects, there were 12 screen fails and 7 withdrawals (5 withdrew pre-infusion). The safety population comprised of 135 subjects, 44 in the placebo group, 45 in the low dose group, and 46 in the high dose group. The ITT population comprised of 132 subjects, 44 in the placebo group, 42 in the low dose group, and 46 in the high dose group. The MITT population comprised of 121 total subjects, 41 in the placebo group, 39 in the low dose group, and 41 in the high dose group. The demographics, baseline characteristics, AKI risk factors, and EuroSCORE, of the subjects in the MITT population can be seen in Tables 1-4.

TABLE 1
MITT Population Demographics

| | | | Low | | High | | Combined | |
|---|---|---|---|---|---|---|---|---|
| | Placebo | | Dose | | Dose | | RBT-1 | |
| | (N = 41) | | (N = 39) | | (N = 41) | | (N = 80) | |
| Mean Age (yrs) | 65.37 | | 64.59 | | 66.59 | | 65.61 | |
| Sex | | | | | | | | |
| Female, N (%) | 11 | (26.8) | 11 | (28.2) | 9 | (22.0) | 20 | (25.0) |
| Male, N (%) | 30 | (73.2) | 28 | (71.8) | 32 | (78.0) | 60 | (75.0) |
| Race | | | | | | | | |
| American Indian | 0 | | 0 | | 1 | (2.4) | 1 | (1.3) |
| or Alaska Native, | | | | | | | | |
| N (%) | | | | | | | | |
| Black, N (%) | 2 | (4.9) | 4 | (10.3) | 1 | (2.4) | 5 | (6.3) |
| Asian, N (%) | 1 | (2.4) | 1 | (2.6) | 2 | (4.9) | 3 | (3.8) |
| White, N (%) | 38 | (92.7) | 32 | (82.1) | 37 | (90.2) | 69 | (86.3) |

| | | | | | | |
|---|---|---|---|---|---|---|
| Other, N (%) | 0 | 2 | (5.1) | 0 | 2 | (2.5) |

| | | | | |
|---|---|---|---|---|
| Weight (kg), | 88.7 | 97.4 | 90.9 | 94.0 |
| Mean (min, max) | (64, 132) | (51, 142) | (57, 150) | (51, 150) |
| BMI (kg/m²), | 29.7 | 32.8 | 30.2 | 31.4 |
| Mean (min, max) | (19, 45) | (18, 48) | (20, 49) | (18, 49) |

TABLE 2
Baseline Characteristics

| | | Low | High | Combined |
|---|---|---|---|---|
| | Placebo | Dose | Dose | RBT-1 |
| | (N = 41) | (N = 39) | (N = 41) | (N = 80) |
| Time of Infusion | | | | |
| Before Surgery | | | | |
| N | 41 | 39 | 41 | 80 |
| Mean (hrs) | 38.6 | 38.6 | 38.4 | 38.5 |
| Surgery Type | | | | |
| CABG Alone, N (%) | 20 (48.8) | 20 (51.3) | 24 (58.4) | 44 (55.0) |
| Valve Alone, N (%) | 8 (19.5) | 13 (33.3) | 9 (22.0) | 22 (27.5) |
| CABG + Valve, N (%) | 13 (31.7) | 6 (15.4) | 8 (19.5) | 14 (17.5) |
| Duration of Surgery | | | | |
| N | 41 | 39 | 41 | 80 |
| Mean (hrs) | 4.941 | 5.051 | 4.929 | 4.988 |
| Time on Pump | | | 41 | 80 |
| N | 41 | 39 | | |
| Mean (hrs) | 1.946 | 1.952 | 1.996 | 1.974 |

TABLE 3
AKI Risk Factors

| | | | | | | | Combined | |
|---|---|---|---|---|---|---|---|---|
| | Placebo | | Low Dose | | High Dose | | RBT-1 | |
| Risk Factor - N (%) | (N = 41) | | (N = 39) | | (N = 41) | | (N = 80) | |
| 1. Combined CABG and valve surgery | 13 | (31.7) | 6 | (15.4) | 8 | (19.5) | 14 | (17.5) |
| 2. Previous cardiac surgery with | 0 | | 1 | (2.6) | 1 | (2.4) | 2 | (2.5) |
| sternotomy | | | | | | | | |
| 3. Documented heart failure (NHYA III/IV) | 2 | (4.9) | 3 | (7.7) | 5 | (12.2) | 8 | (10) |
| within 1 year prior to surgery | | | | | | | | |
| 4. LVEF ≤35% | 2 | (4.9) | 3 | (7.7) | 4 | (9.8) | 7 | (8.8) |
| 5. Congestive heart failure | 6 | (14.6) | 5 | (12.8) | 7 | (17.1) | 12 | (15) |
| 6. Diabetes mellitus requiring insulin | 3 | (7.3) | 6 | (15.4) | 8 | (19.5) | 14 | (17.5) |
| 7. T2D with albuminuria (urine albumin | 0 | | 0 | | 0 | | 0 | |
| >300 mg/g of creatinine, as documented | | | | | | | | |
| in medical history) | | | | | | | | |
| 8. Pre-operative anemia (hemoglobin | 1 | (2.4) | 1 | (2.6) | 0 | | 1 | (1.3) |
| <10 g/dL upon screening) | | | | | | | | |
| 9. Currently hospitalized for management | 8 | (19.5) | 5 | (12.8) | 12 | (29.3) | 17 | (21.3) |
| of cardiac or pulmonary disease | | | | | | | | |
| 10. eGFR ≥20 to <30 mL/min/1.73 m² | 2 | (4.9) | 0 | | 0 | | 0 | |
| 11. eGFR ≥30 to <60 mL/min/1.73 m² | 4 | (9.8) | 13 | (33.3) | 13 | (31.7) | 26 | (32.5) |
| 12. Age ≥65 years | 23 | (56.1) | 23 | (59.0) | 25 | (61.0) | 48 | (60.0) |

TABLE 4
EuroSCORE

| | | | | Combined |
|---|---|---|---|---|
| | Placebo | Low Dose | High Dose | RBT-1 |
| EuroSCORE | (N = 41) | (N = 39) | (N = 41) | (N = 80) |
| Mean | 1.89 | 2.78 | 2.39 | 2.58 |
| (Min, Max) | (0.56, 9.73) | (0.50, 17.35) | (0.50, 9.13) | (0.50, 17.35) |
| Median | 1.47 | 1.05 | 1.93 | 1.52 |
| Low Risk | 37 (90) | 30 (77) | 30 (73) | 60 (75) |
| (<2.99), N (%) | | | | |
| Medium Risk | 2 (5) | 4 (10) | 9 (22) | 13 (16) |
| (3 to 5.99), | | | | |
| N (%) | | | | |
| High Risk | 2 (5) | 5 (13) | 2 (5) | 7 (9) |
| (>5.99), N (%) | | | | |

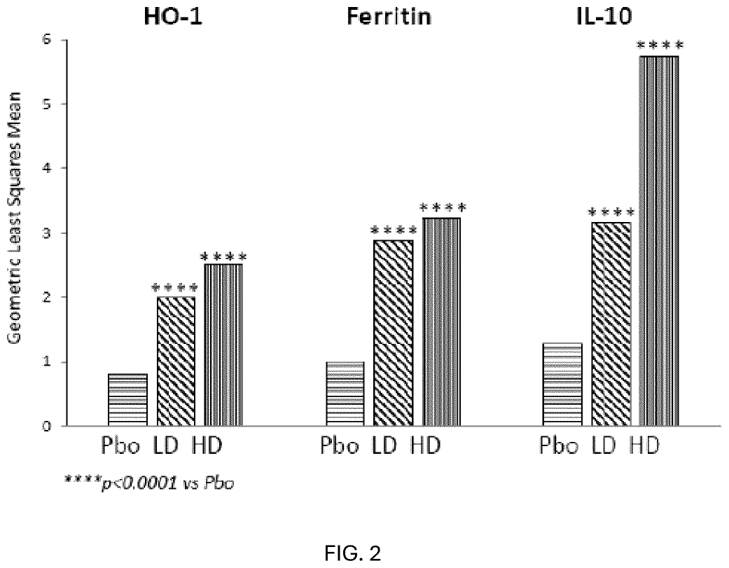

FIG. 2 shows a graph demonstrating the statistically significant increase in anti-inflammatory and antioxidant biomarkers of cytoprotective preconditioning with RBT-1 for the MITT population (p<0.0001). The biomarkers assessed were interleukin-10 (IL-10), heme oxygenase-1 (HO-1), and ferritin. The composite biomarker response for the data shown in FIG. 2 is shown in Table 5.

TABLE 5
Composite Biomarker Response

| | Placebo | Low Dose | High Dose |
|---|---|---|---|
| | (N = 44) | (N = 42) | (N = 46) |
| Mean | 0.98 | 2.65 | 3.62 |
| P-value vs Pbo | | <0.0001 | <0.0001 |
| P-value LD vs HD | | | 0.0046 |

All of the subsequent analyses have been conducted on the MITT population as predefined.

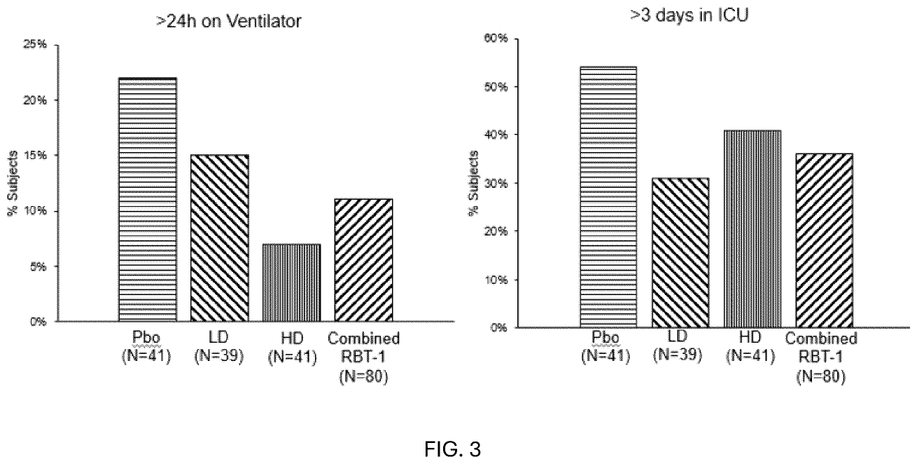

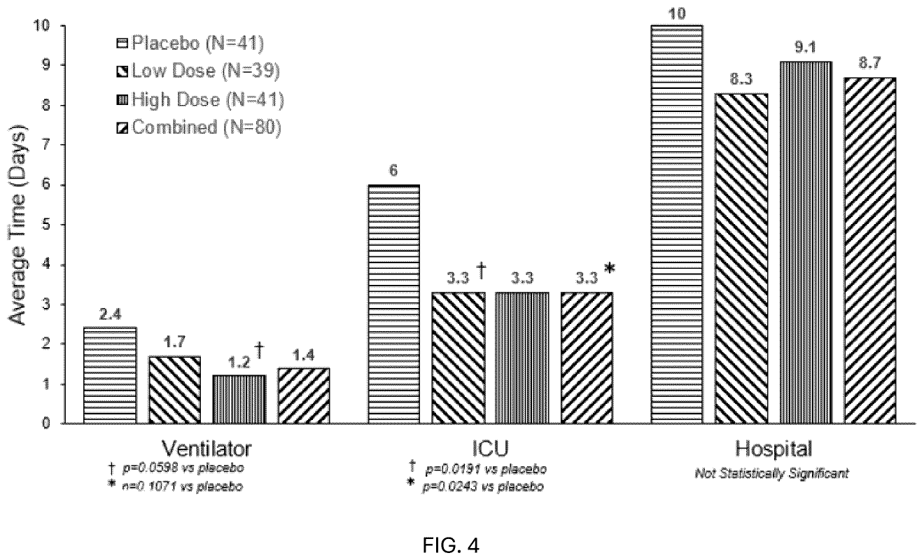

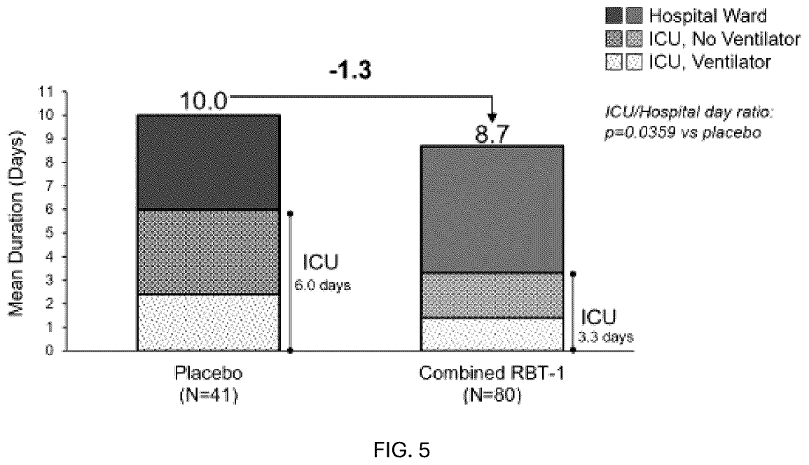

FIG. 3 shows a graph of the extended time on the ventilator and in the ICU spent by subjects in each group. The graph illustrates a decline in the percentage of subjects that required a ventilator post-surgery between the placebo group and the low and high dose groups with the high dose group having the lowest percentage. FIG. 3 additionally shows a decline in the percentage of subjects that required greater than 3 days in the ICU post-surgery with the low dose group having the smallest percentage. FIG. 4 shows a graph of the average time of subjects in each group spent on a ventilator, in the ICU, and in the hospital. The graph shows a reduction in ventilator, ICU, and hospital time in patients treated with RBT-1. An overall improvement in mean composition of hospitalization in patients treated with RBT-1 can be seen in FIG. 5.

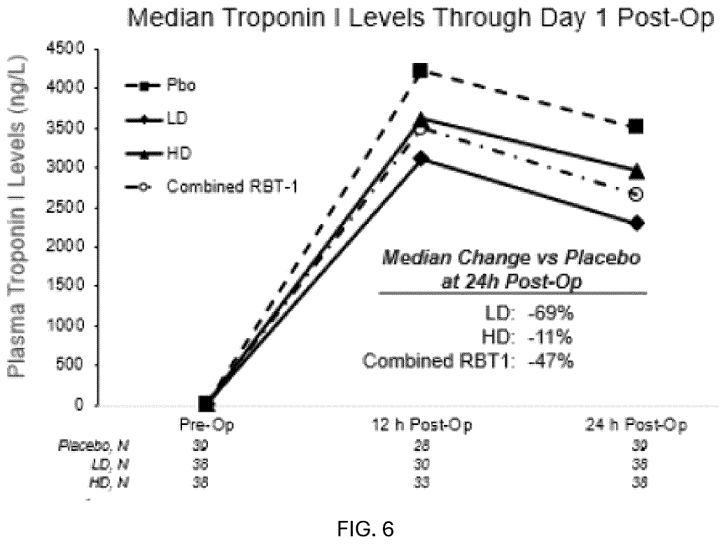

The low dose of RBT-1 group exhibited the lowest median levels of Troponin I levels while the high dose group additionally exhibited lower levels than that of the placebo group. There was a clinically meaningful reduction in Troponin I levels (47%) for the combined RBT-1 groups as compared to the placebo group. FIG. 6 shows the increase in plasma troponin I from pre-operative baseline to 12 hours (left series) and 1 day (right series) after cardiac surgery. The analysis population, derived from the modified intent-to treat population, excluded patients who had undergone mitral valve repair or replacement, ablation, or septal myectomy due to the expected significant increase in troponin I levels following these major surgeries.

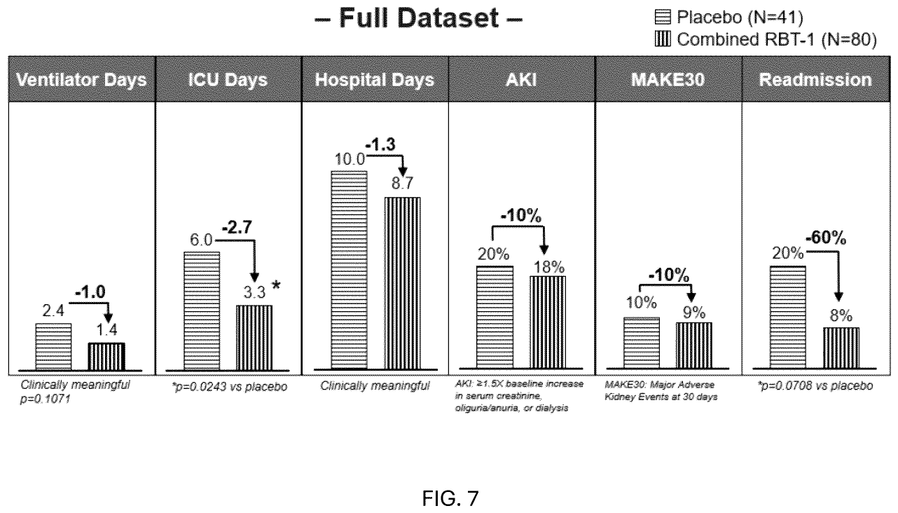

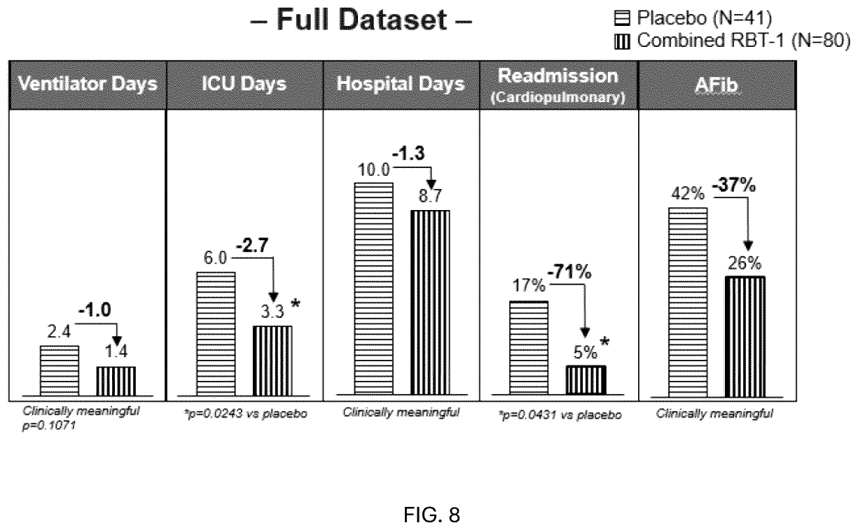

In FIG. 7 and FIG. 8, graphs illustrate the improvement of postoperative outcomes of the combined RBT-1 groups over the placebo group in measurements such as ventilator days, ICU days, hospital days, AKI, major adverse kidney events at 30 days (MAKE30), readmission, readmission (cardiopulmonary), and Atrial Fibrillation (AFib). There is a clinically meaningful improvement in subjects treated with RBT-1 over the placebo in ventilator days (−1 day), length of hospital stay (−1.3 days), AKI rate (−10%), and in AFib rates (−37%).

There was a statistically significant (−2.7 days, p<0.03) reduction in days in the ICU. A statistically significant (−71%, p<0.05) reduction in 30-day hospital readmission rates due to cardiopulmonary causes and a 60% reduction in all-cause readmissions. The data also indicated a generally well-tolerated safety profile.

A win ratio for the combination of high and low doses of RBT-1 was calculated on the data collected in the trial. A win ratio is a method for examining composite endpoints and has since been widely adopted in cardiovascular (CV) trials. Improving upon conventional methods for analyzing composite endpoints, the win ratio accounts for relative priorities of the components and allows the components to be different types of outcomes. The win ratio is further described by Redfors B. et al. The win ratio approach for composite endpoints: practical guidance based on previous experience. Eur Heart J. 2020 Dec. 7; 41(46):4391-4399. doi: 10.1093/eurheartj/chaa665. PMID: 32901285.

Table 6-9 illustrate the win ratio calculation for the trial. In an analysis of composite endpoint of death, ICU days, ventilator days, atrial fibrillation rates, hospital days and hospital admission rates using the win ratio method, a highly statistically significant benefit was observed among the treated groups (win ratio 1.63, p<0.02). These positive topline data provide strong support for RBT-1's potential to reduce the risk of multiorgan injury and thereby improve post-operative outcomes in patients undergoing cardiothoracic surgery. These data suggest that RBT-1 can provide wide protection against organ damage, and has the potential to reduce post-operative complications, lengths of stay, and costs of care.

TABLE 6

| | HD + LD | Placebo | p-value (2-sided) |
|---|---|---|---|
| Death (%) | 3.75 | 7.317073171 | 0.392277603 |
| ICU Days (mean) | 3.2875 | 6 | 0.022492274 |
| Vent Days (mean) | 1.4375 | 2.43902439 | 0.104440839 |
| Afib (%) | 26.25 | 41.46341463 | 0.087901013 |
| Readmission (%) | 10 | 24.3902439 | 0.035253013 |
| Hosp Days (mean) | 8.7 | 9.975609756 | 0.800942124 |

TABLE 7

| | Died | ICU | Vent | AFib | Readmission | Hosp Days |
|---|---|---|---|---|---|---|
| Win | 231 | 1554 | 26 | 104 | 43 | 55 |
| Loss | 114 | 932 | 13 | 69 | 17 | 87 |

TABLE 8

| | Win | Tie | Loss |
|---|---|---|---|
| Pairs | 2013 | 35 | 1232 |

TABLE 9

| | Win Ratio | 1-sided p-value |
|---|---|---|
| Result | 1.63392857 | 0.0157 |

Several treatment comparisons stratified by the weight category of subjects were done to determine benefits of varying per-kg dosing (mg/kg) and to explore a minimum viable dose. Unexpectedly, the drug benefit increased at lower doses. In general, the ideal range is 20-70 mg based on a 70 kg patient, preferably 28-63 mg, or 35-63 mg.

To define the categories, tertials were used (i.e., about ⅓ of the patients in each category). “Low weight” is less than about 81 kg (mean of 72 kg), “medium weight” is between about 81 kg and 99 kg (mean of 90 kg) and “high weight” is greater than about 99 kg (mean of 115 kg). Table 10 shown below shows the win-ratio results stratified by the weight category. Using the mean weights within categories, the results displayed in order of per-kg dosing show more of a U-shaped relationship. Once a certain threshold of dosage is crossed, the effectiveness of the dose declines and the benefits of the dose declines.

TABLE 10
Win-Ratio

| Comparison | Weight Category | Dose (mg/kg) | Win-ratio | 95% CI |
|---|---|---|---|---|
| LD vs Placebo | High | 0.4 | 2.0 | 0.8, 5.1 |
| LD vs Placebo | Medium | 0.5 | 1.6 | 0.6, 4.30 |
| LD vs Placebo | Low | 0.6 | 2.5 | 0.8, 7.4 |
| HD vs Placebo | High | 0.8 | 1.8 | 0.7, 5.0 |
| HD vs Placebo | Medium | 1.0 | 1.4 | 0.5, 3.8 |
| HD vs Placebo | Low | 1.3 | 1.0 | 0.3, 3.0 |

The 95% confidence interval (CI) results are rough estimates to illustrate that these subgroup analyses have a small sample size, and the Win-Ratios aren't very precise. However, these results overall suggest a peak effect around 0.6 or 0.7 mg/kg. For example, a 100 kg person that corresponds to a suggested dosing of 63 mg, although the data also suggests that 63 mg might be a bit too high for a lighter patient. In one embodiment, the dose administered to the patient is a function of the patient's weight to improve the benefits of the dose.

Adverse events of interest in the trial were recorded. An overview of treatment-emergent adverse events (TAEs) are shown in Table 11 and TAEs of interest for the safety population are shown in Table 12.

TABLE 11
An overview of Treatment-Emergent Adverse Events (TEAEs)

| | | | | | | | Combined | |
|---|---|---|---|---|---|---|---|---|
| | Placebo | | LD | | HD | | RBT-1 | |
| | (N = 44) | | (N = 45) | | (N = 46) | | (N = 91) | |
| Subjects with any TEAE | 40 | (90.9) | 39 | (86.7) | 44 | (95.7) | 83 | (91.2) |
| Maximum Severity of Mild | 7 | (15.9) | 11 | (24.4) | 15 | (32.6) | 36 | (28.6) |
| Maximum Severity of | 18 | (40.9) | 17 | (37.8) | 17 | (37.0) | 34 | (37.4) |
| Moderate | | | | | | | | |
| Maximum Severity of | 15 | (34.1) | 11 | (24.4) | 12 | (26.1) | 23 | (25.3) |
| Severe | | | | | | | | |
| Subjects with at least one | 6 | (13.6) | 12 | (26.7) | 18 | (39.1) | 30 | (33.0) |
| Treatment-Related TEAE | | | | | | | | |
| Excluding Adjudicated | 5 | (11.4) | 7 | (15.6) | 8 | (17.4) | 15 | (16.5) |
| Photosensitivity | | | | | | | | |
| Subjects with at least one | 18 | (40.9) | 13 | (28.9) | 22 | (47.8) | 35 | (38.5) |
| Serious TEAE | | | | | | | | |
| Subjects Discontinued due | 0 | | 0 | | 0 | | 0 | |
| to TEAE | | | | | | | | |
| Died on Study | 3 | (6.8) | 1 | (2.2) | 2 | (4.3) | 3 | (3.3) |

| | | | |
|---|---|---|---|
| Cause of Deaths | Sepsis | Acute | Cardiogenic shock |
| | Stroke | respiratory | CO₂ retention from |
| | Cardiac | failure | chronic lung disease |
| | arrest | | |

TABLE 12
TEAEs of Interest - Safety Population

| | | | | | | | Combined | |
|---|---|---|---|---|---|---|---|---|
| | Placebo | | LD | | HD | | RBT-1 | |
| | (N = 44) | | (N = 45) | | (N = 46) | | (N = 91) | |
| Atrial Fibrillation, | 17 | (38.6) | 11 | (24.4) | 10 | (21.7) | 21 | (23.1) |
| N (%) | | | | | | | | |
| Anemia, N (%) | 11 | (25.0) | 8 | (17.8) | 6 | (13.0) | 14 | (15.4) |
| Hypervolemia, N (%) | 10 | (22.7) | 4 | (8.9) | 5 | (10.9) | 9 | (9.9) |
| Leukocytosis, N (%) | 6 | (13.6) | 3 | (6.7) | 4 | (8.7) | 7 | (7.7) |

Occurrences of photosensitivity were recorded and are shown in Table 13. Three surgeries were postponed due to photosensitivity of the subjects. Each postponed surgery due to photosensitivity occurred for high dose subjects and were additionally exposed to the sun for a prolonged period of time post-infusion.

TABLE 13
Photosensitivity AEs Considered Related by Site

| | Placebo | LD | HD |
|---|---|---|---|
| | (N = 44) | (N = 45) | (N = 46) |
| Photosensitivity, N (%) | 1 (2) | 5 (11) | 10 (22) |
| Day of Onset Post-Infusion, Median | 14.0 | 2.0 | 2.0 |
| Day of Onset Post-Infusion, Median | — | 4.0 | 8.0 |

### Example 2

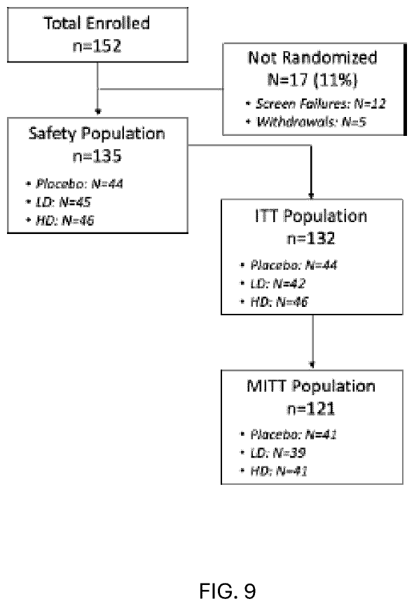

152 patients were enrolled at 19 sites across the US, Canada, and Australia and 135 patients were randomized. The safety population consisted of patients, all of whom received study drug. Of those, 132 (98%) patients had biomarker measurements collected (ITT population). From the ITT population, 121 patients had surgery on time (24 to 48 hours after infusion) and constituted the MITT population, for whom secondary endpoints and clinical outcomes were evaluated. FIG. 9 shows enrollment and patient populations. The total number of patients enrolled and randomized are shown. Distribution of patients by dose group are provided for each patient population. Reasons for screen failures included high serum ferritin (N=5), receipt of IV iron prior to planned infusion (N=1), acute organ injury/unstable organ function (n=4), hypersensitivity to tin-based products (n=1), and change in surgery location (n=1). Five patients withdrew from the study prior to treatment; two patients withdrew from the study post-treatment (one due to postponed surgery and one due to difficulty with blood draws).

In the MITT population, 41 patients received placebo (normal saline), 39 patients received low-dose RBT-1 (45 mg SnPP/240 mg FeS), and 41 patients received high-dose RBT-1 (90 mg SnPP/240 mg FeS). Baseline characteristics of the randomized patients were similar among groups (Table 14). Time between infusion and start of surgery, as well as time on CPB, were also similar between the three groups.

TABLE 14

| | Placebo | | Low-dose | | High-dose | |
|---|---|---|---|---|---|---|
| | (N = 41) | | (N = 39) | | (N = 41) | |
| Age (years), | 65 | (19, 81) | 65 | (46, 82) | 67 | (37, 86) |
| Mean (min, max) | | | | | | |
| Sex Male, N (%) | 30 | (73) | 28 | (72) | 32 | (78) |
| Race, N (%) | | | | | | |
| American Indian | 0 | | 0 | | 1 | (2) |
| Black | 2 | (5) | 4 | (10) | 1 | (2) |
| Asian | 1 | (2) | 1 | (3) | 2 | (5) |
| White | 38 | (93) | 32 | (82) | 37 | 37(90) |
| Other | 0 | | 2 | (5) | 0 | |
| Weight (kg), | 89 | (64, 132) | 98 | (51, 142) | 91 | (57, 150) |
| Mean (min, max) | | | | | | |
| BMI (kg/m2), | 30 | (19, 45) | 33 | (18, 48) | 30 | (20, 49) |
| Mean (min, max) | | | | | | |
| EuroSCORE II, | 2.1 | (1, 10) | 2.8 | (1, 17) | 2.4 | (1, 9) |
| Mean (min, max) | | | | | | |
| Low Risk (<3), | 35 | (85) | 31 | (80) | 31 | (76) |
| N (%) | | | | | | |
| Medium Risk | 4 | (10) | 3 | (8) | 8 | (20) |
| (3 to 6), N (%) | | | | | | |
| High Risk (≥6), | 2 | (5) | 5 | (13) | 2 | (5) |
| N (%) | | | | | | |
| ≥3 AKI Risk | 39 ± 9.9 | | 39 ± 9.2 | | 38 ± 9.4 | |
| Factors, * N (%) | | | | | | |
| Time infusion to | | | | | | |
| surgery, Mean ± SD | | | | | | |
| (hrs) | | | | | | |
| Surgery Type | | | | | | |
| CABG alone, N (%) | 20 | (49) | 20 | (51) | 24 | (59) |
| Valve alone, N (%) | 7 | (17) | 13 | (33) | 9 | (22) |
| CABG + Valve, | 14 | (34) | 6 | (15) | 8 | (20) |
| N (%) | | | | | | |

| | | | |
|---|---|---|---|
| Time on CPB, | 2.0 ± 1.0 | 2.0 ± 0.8 | 2.0 ± 1.2 |
| Mean ± SD (hrs) | | | |

### Primary Outcome

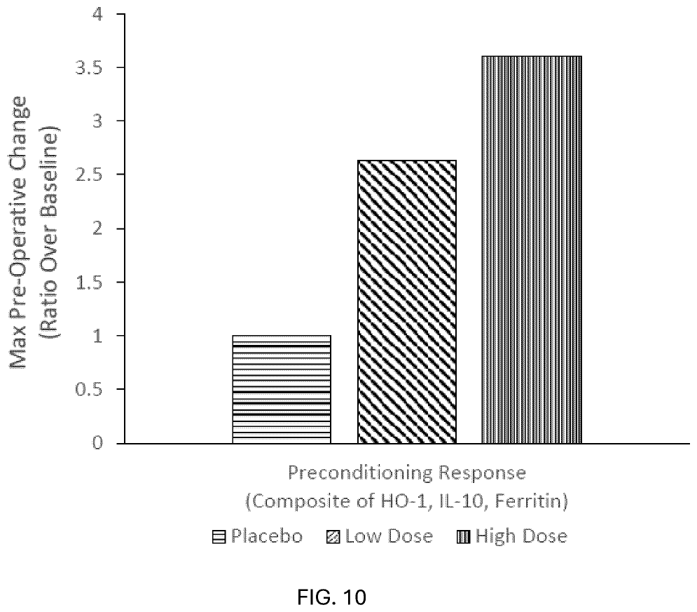

The mean composite of the maximum pre-operative change from baseline in HO-1, IL-10, and ferritin was 1.00 (95% CI: 0.86, 1.17) in the placebo group, 2.63 (95% CI: 2.25, 3.07) in the low dose RBT-1 group, and 3.60 (95% CI: 3.10, 4.18) in the high-dose RBT-1 group, FIG. 10, p<0.0001 for both comparisons. FIG. 10 shows the primary endpoint—preconditioning response. The preconditioning response is shown as the geometric least squares mean for the ratio of the maximum pre-operative change over baseline in a composite of heme oxygenase-1 (HO-1), interleukin-10 (IL-10), and ferritin; *** p<0.001 vs placebo.

### Secondary Endpoints

AKI incidence and sustained reduction in urine output were numerically lower with both doses of RBT-1 but were not significantly different compared with placebo (Table 15). There were no significant changes in the composite of renal tubular injury biomarkers (Table 15), and these biomarkers did not correlate with the maximum change in serum creatinine.

TABLE 15

| | Placebo | Low-dose | P value | High-dose | P value |
|---|---|---|---|---|---|
| | (N = 41) | (N = 39) | (vs. placebo) | (N = 41) | (vs. placebo) |
| AKI (%) ** | 8 (19.5) | 7 (17.9) | >0.999 | 7 (17.1) | >0.999 |
| Sustained reduction | 4 (9.8) | 2 (5.1) | 0.676 | 2 (4.9) | 0.676 |
| urine output, N(%) *** | | | | | |
| Tubular injury | 6.10 | 10.84 | 0.068 | 7.89 | 0.395 |
| biomarker response, | (3.96, 9.39) | (6.92, 16.98) | | (5.09, 12.23) | |
| GLSM* (95% CI) | | | | | |

*GLSM, geometric least squares mean of the ratio of max Post-Op value over Baseline.
**AKI is defined as a ≥1.5 × serum creatinine increase from baseline, sustained reduction in urinary output, or initiation of dialysis post-cardiac surgery through Day 5.
***Sustained reduction in urinary output was defined as a reported adverse event (AE) of oliguria, anuria, or “sustained” reduction in urine output post-cardiac surgery through Day 5.

### Other Prespecified Endpoints

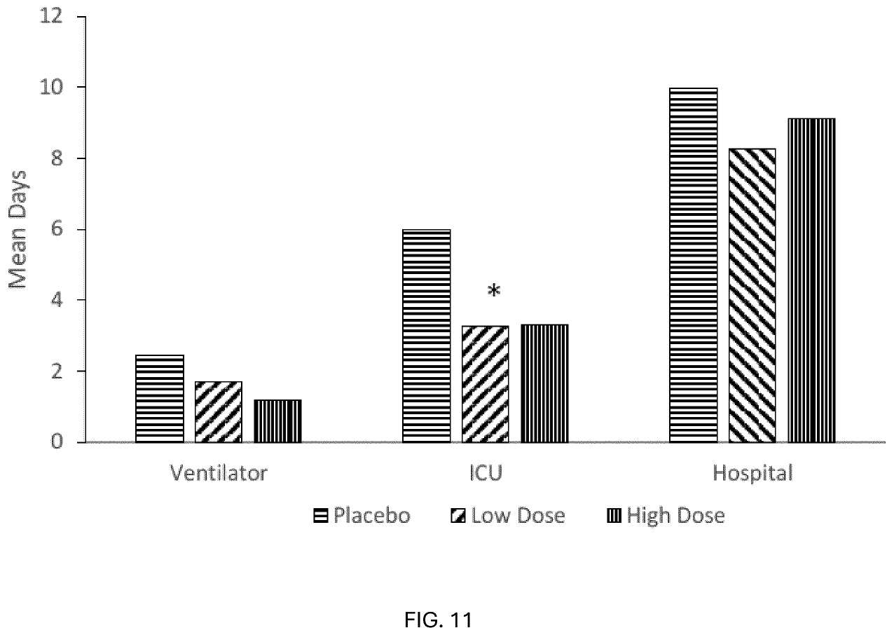

Hospitalization course was assessed by time on ventilator and length of stay (LOS) in the ICU and hospital. FIG. 11 shows Ventilator, ICU, and Hospital Course. The mean time (days) on ventilator, in the ICU, and in the hospital is shown for each treatment group; *p=0.02 vs placebo. The mean time on ventilator was 2.4 days, 1.7 days, and 1.2 days in the placebo, low-dose RBT-1, and high-dose RBT-1 groups (p=0.428 and p-0.060 vs placebo, respectively). The mean LOS in ICU was 6 days in the placebo group and 3.3 days in the low dose and high-dose RBT-1 groups (p=0.019 and p=0.128 vs placebo, respectively). The mean LOS in hospital was 10.0 days, 8.3 days, and 9.1 days in the placebo, low-dose RBT-1, and high dose RBT-1 groups, respectively (p-0.744 and p-0.918 vs placebo, respectively).

Post-operative complications included three deaths (7.3%) in the placebo group, one death (2.6%) in the low-dose RBT-1 group and two deaths (4.9%) in the high-dose RBT-1 group (Table 16). Dialysis for AKI was needed in one patient (2.4%) in the placebo group but none in the RBT-1 groups. Post-operative atrial fibrillation developed in 17 (42%), 12 (31%), and 10 (24%) patients in the placebo, low-dose RBT-1, and high-dose RBT-1 groups, respectively (Table 16). Hypervolemia was diagnosed in 10 (24%), 3 (8%), and 4 (10%) of patients in the placebo, low-dose RBT-1, and high-dose RBT-1 groups (Table 16). Of note, the change from baseline in post-operative troponin I levels 1-day post-surgery was reduced by 63% in the low dose RBT-1 group and 30% in the high-dose RBT-1 group (p=0.016 for low-dose RBT-1 vs placebo). Patients were evaluated for MAKE at 30, 60, and 90 days. The number of patients with MAKE was low as expected with this unenriched population, and no statistical difference between groups were observed (Table 16).

Finally, seven patients (17%) were readmitted to the hospital at 30-days post-discharge for a cardiopulmonary diagnosis in the placebo group compared with two patients (5%) each in the low-dose and high dose RBT-1 groups (Table 16). At 60- and 90-days post-discharge, eight patients (21%) in the placebo group required cardiopulmonary readmissions in contrast to two patients (5%) each in the low-dose and high-dose RBT-1 groups. All-cause readmissions showed similar results (Table 16).

TABLE 16

| | | Low- | RR | P value | High- | RR | P value |
|---|---|---|---|---|---|---|---|
| | Placebo | dose | 95% | (vs. | dose | 95% | (vs. |
| | (N = 41) | (N = 39) | CI | placebo) | (N = 41) | CI | placebo) |
| Death, N (%) | 3(7.3) | 1 (2.6) | | | 2 (4.9) | | |
| AKI with dialysis, | 1 (2.4) | 0 | | | 0 | | |
| N (%) | | | | | | | |
| MAKE 30, N (%) | 4(9.8) | 1(2.6) | | | 4(9.8) | | |
| MAKE 60, N (%)¶ | 2(5.0) | 1(2.6) | | | 3(7.3) | | |
| Atrial fibrillation, | 17 | 12 | 0.74 | 0.359 | 10(24.4) | 0.59 | 0.158 |
| N (%) | (41.5) | (30.8) | (0.36, | | | (0.28, | |
| | | | 1.36) | | | 1.13) | |
| Hypervolemia, N (%) | 10(24.4) | 3(7.7) | 0.32 | 0.067 | 4(9.8) | 0.4 | 0.141 |
| | | | (0.07, | | | (0.07, | |
| | | | 1.01) | | | 1.15) | |
| 30 days | 7(18.4) | 2(5.3) | 0.29 | 0.153 | 2(5.1) | 0.28 | 0.087 |
| Cardiopulmonary | | | (0.03, | | | (0.03, | |
| readmissions, N (%)*‡ | | | 1.00) | | | 1.14) | |
| 30 days | 8 (21.1) | 2 (5.3) | 0.25 | 0.086 | 4(10.3) | 0.49 | 0.087 |
| All readmissions, | | | (0.03, | | | (0.08, | |
| N (%)*‡ | | | 1.00) | | | 1.53) | |
| 60 Cardiopulmonary | 8 (21.1) | 2 (5.3) | 0.25 | 0.086 | 2(5.1) | 0.24 | 0.047 |
| readmissions, | | | (0.03, | | | (0.03, | |
| N (%)*‡ | | | 1.00) | | | 0.98) | |
| 60 days all | 10 | 3 (7.9) | 0.30 | 0.065 | 5(12.8) | 0.49 | 0.160 |
| readmissions, | (26.3) | | (0.07, | | | (0.13, | |
| N (%)*‡ | | | 0.96) | | | 1.29) | |

¶MAKE 60 and 90 results were identical.
*Subjects who died during index hospitalization are excluded from readmission analysis; total sample size for placebo, LD, and HD is 38, 38, and 39 patients, respectively.
‡Cardiopulmonary readmissions and All readmissions results were identical at 60 days and 90 295 days.

### Post Hoc Analysis

Given the suggested multi-organ benefit of RBT-1, we explored the effects of RBT-1 in a post hoc composite analysis (win ratio) wherein clinical outcomes were assessed in rank order of severity (death, AKI requiring dialysis, ICU days, 30-day cardiopulmonary readmission, atrial fibrillation, and hospital days). The win ratio was 2.02 (p=0.004) in the low-dose RBT-1 group and 1.34 (p-0.131) in the high-dose RBT-1 group (Table 17 A-B), showing that patients treated with RBT-1 had improved outcomes compared to those treated with placebo.

TABLE 17 A

| | | | | Win Ratio | |
|---|---|---|---|---|---|
| | Wins | Tie | Loss | (95% CI) | p-value |
| Low dose RBT-1 | 1056 | 21 | 522 | 2.02 (1.19, 3.44) | 0.004 |
| High dose RBT-1 | 952 | 16 | 713 | 1.34 (0.80, 2.23) | 0.131 |

TABLE 17 B

| | | AKI | | 30-Day | | |
|---|---|---|---|---|---|---|
| | | Requiring | ICU | Cardiopulmonary | Atrial | Hospital |
| | Death | Dialysis | Days | Readmission | Fibrillation | LOS |
| Low-dose RBT-1 | | | | | | |
| Win | 114 | 1 | 817 | 50 | 38 | 46 |
| Loss | 38 | 0 | 388 | 8 | 42 | 46 |
| High-dose RBT-1 | | | | | | |
| Win | 117 | 2 | 734 | 34 | 40 | 25 |
| Loss | 76 | 0 | 544 | 18 | 21 | 54 |

Win ratio, based on the Finkelstein-Schoenfeld method, derived from rank order analysis of death, AKI requiring dialysis, ICU days, 30-day cardiopulmonary readmission, atrial fibrillation, and hospital length of stay and comparisons made between each patient on placebo and each patient on low-dose RBT-1 and high-dose RBT-1. The cells in the upper table indicate the overall number of wins and losses across all endpoints, and the cells in the lower table indicate the number of wins and losses observed at each endpoint in the hierarchy.

### Safety Outcomes

RBT-1 was well tolerated, with the primary adverse event (AE) considered related to RBT-1 being photosensitivity, which is a known reaction to the SnPP component of RBT-1. Photosensitivity was dose-dependent, occurring in 6 of 45 patients (13%) treated with low-dose RBT-1 and in 12 of 46 patients (26%) treated with high-dose RBT-1. In general, photosensitivity reactions were transient and mild to moderate in intensity. The median time of onset of photosensitivity was 2.5 days in the low-dose RBT-1 group and 2.0 days in the high-dose RBT-1 group; median time to resolution was 3.5 days in the low-dose RBT-1 group and 7.0 days in the high-dose RBT-1 group. All photosensitivity reactions resolved within 28 days in the LD group and within 93 days in the HD group. Three photosensitivity reactions in the high-dose RBT-1 group resulted in delayed surgery.

### DISCUSSION

This study of RBT-1 met its primary endpoint, demonstrating a statistically significant increase in the levels of cytoprotective proteins (plasma HO-1, IL-10, and ferritin), which are surrogate measures for RBT-1-mediated activation of a preconditioning response. The overall incidence of AKI was low and AKI-related outcomes did not show statistical significance in this unenriched population.

Cardiac surgery especially with cardiopulmonary bypass induces systemic inflammation which can lead to multi-organ dysfunction, impacting clinical outcomes. Importantly, inflammation and oxidative stress exist in a feed-forward loop, magnifying the response of each pathway. The detrimental effect of these cell-damaging mediators can be seen in the phenomenon of “organ crosstalk,” wherein damage in one organ leads to damage in another organ. Just as these harmful mediators can adversely impact distant organs, cytoprotective mediators can also be carried from one organ to another.

The benefits observed with RBT-1 are likely related to mitigation of these adverse effects by activating anti-inflammatory and antioxidant pathways prior to surgery, hence resulting in direct and indirect beneficial effects in various organs. These broad organ protective benefits may result in an improvement in clinical outcomes as manifested by reduced time on ventilator, need for vasopressors, new-onset atrial fibrillation and fluid overload in the short term, and a decrease in cardiopulmonary hospital readmissions in the longer term. To further assess this hypothesis, we explored the effects of RBT-1 in a post hoc analysis using a win ratio based composite endpoint wherein clinical outcomes were assessed in rank order of severity. This assessment day cardiopulmonary readmission, atrial fibrillation, and hospital days suggests clinical improvement in response to RBT-1, which will be confirmed in an upcoming larger study.

The safety profile of RBT-1 showed that it was well tolerated, with the primary drug-related adverse effect being photosensitivity, which was dose-related and time-limited. The SnPP component (a metalloporphyrin) of RBT-1 is likely the cause of photosensitivity as metalloporphyrins are light responsive and may lead to a sunburn in patients exposed to the sun, especially if sun exposure is prolonged or sunscreen is not used.

Given the better safety profile of low-dose RBT-1 (45 mg SnPP/240 mg FeS) with respect to photosensitivity, this dose was selected for the Phase 3 study, wherein the planned primary endpoint will be a hierarchical composite of clinical outcomes in rank order of severity.

### Example 3

152 patients were enrolled at 19 sites across the US, Canada, and Australia (Table 18)

TABLE 18

| | High-dose | | Low-dose | | Placebo | |
|---|---|---|---|---|---|---|
| | (N = 46) | | (N = 42) | | (N = 44) | |
| Age (years), Mean (min, max) | 66.0 ± 11.4 | | 64.2 ± 8.6 | | 65.7 ± 10.7 | |
| Sex | | | | | | |
| Female | 10 | (22%) | 11 | (26%) | 12 | (27%) |
| Male | 36 | (78%) | 31 | (74%) | 32 | (73%) |
| Race | | | | | | |
| White | 41 | (89%) | 35 | (83%) | 41 | (93%) |
| Black | 2 | (4%) | 4 | (10%) | 2 | (5%) |
| Asian | 2 | (4%) | 1 | (2%) | 1 | (2%) |
| American Indian | 1 | (2%) | 0 | (0%) | 0 | (0%) |
| Other | 0 | (0%) | 2 | (5%) | 0 | (0%) |
| Weight (kg), Mean (min, max) | 91.1 ± 19.6 | | 97.5 ± 20.9 | | 90.3 ± 18.9 | |
| BMI (kg/m2), Mean (min, max) | 30.3 ± 6.6 | | 32.5 ± 6.3 | | 30.0 ± 5.8 | |
| EuroSCORE II, Mean (min, max) | 1.7 | (1.1-2.7) | 1.2 | (0.9-2.7) | 1.5 | (0.9-2.3) |
| Low Risk (<3), N (%) | 35 | (76%) | 33 | (79%) | 36 | (84%) |
| Medium Risk (3 to 6), N (%) | 9 | (20%) | 4 | (10%) | 5 | (12%) |
| High Risk (≥6), N (%) | 2 | (4%) | 5 | (12%) | 2 | (5%) |
| Acute Kidney Injury Risk Factors | | | | | | |
| Age ≥65 years | 28 | (61%) | 24 | (57%) | 26 | (59%) |
| Diabetes mellitus requiring insulin | 8 | (17%) | 7 | (17%) | 4 | (9%) |
| Congestive heart failure | 7 | (15%) | 6 | (14%) | 7 | (16%) |
| Heart failure (NYHA III/IV) | 6 | (13%) | 3 | (7%) | 4 | (9%) |
| within 1 year prior to surgery | | | | | | |
| Previous cardiac surgery with | 1 | (2%) | 1 | (2%) | 0 | (0%) |
| sternotomy | | | | | | |
| Left ventricular ejection fraction | 5 | (11%) | 6 | (14%) | 7 | (16%) |
| ≤35% | | | | | | |
| Estimated glomerular filtration rate | 13 | (28%) | 13 | (31%) | 8 | (18%) |
| ≥20 to <60 mL/min/1.73 m² | | | | | | |
| Preoperative anemia (hemoglobin | 0 | (0%) | 1 | (2%) | 1 | (2%) |
| <10 g/dL) | | | | | | |
| Hospitalized for management of | 11 | (24%) | 6 | (14%) | 8 | (18%) |
| cardiac or pulmonary disease | | | | | | |
| Time infusion to surgery, | 40.9 | (26.0-44.7) | 40.9 | (28.2-44.2) | 41.3 | (24.8-44.8) |
| Mean ± SD (hrs) | | | | | | |
| Time on cardiopulmonary bypass, | 1.8 | (1.3-2.3) | 1.7 | (1.5-2.4) | 1.6 | (1.3-2.3) |
| median (IQR), h | | | | | | |
| Surgery Type | | | | | | |
| CABG alone, N (%) | 24 | (52%) | 23 | (55%) | 22 | (50%) |
| Valve alone, N (%) | 11 | (24%) | 13 | (31%) | 8 | (18%) |
| CABG + Valve, N (%) | 11 | (24%) | 6 | (14%) | 14 | (32%) |

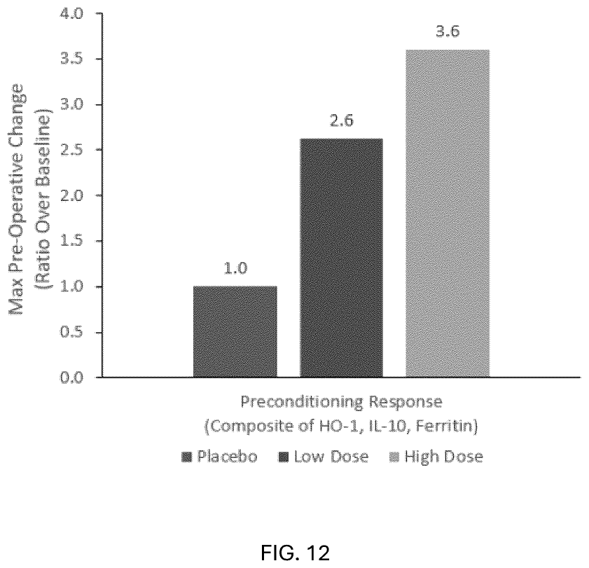

Data arc n (%) unless otherwise specified. Percentages are rounded. CABG=coronary artery bypass grafting. EuroSCORE=European system for cardiac operative risk evaluation. NYHA=New York Heart Association. Among the enrolled patients. 135 were randomly assigned to either the high-dose RBT-1 group (n=46), low-dose RBT-1 group (n=45), or placebo group (n=44). FIG. 12 shows the preconditioning response is shown as the geometric least squares mean for the ratio of the maximum pre-operative change over baseline in a composite of heme oxygenase-1 (HO-1), interleukin-10 (IL-10), and ferritin in the intent-to-treat population. HO=heme oxygenase, IL=interleukin.

The planned interim analysis was conducted from May 9 to Jun. 6, 2022 and included the first 62 patients randomized (n=20 high-dose, n=19 low-dose, n=23 placebo). The interim results indicated significant differences in the primary outcome between both high-dose RBT-1 (p<0.0001) and low-dose RBT-1 (p<0.0001) groups compared with the placebo group (Table 19).

TABLE 19

| | | | | High-dose v. placebo | | Low-dose v. placebo | |
|---|---|---|---|---|---|---|---|
| | High- | Low- | | GLSM | | GLSM | |
| | dose | dose | Placebo | Ratio | | Ratio | |
| | n = 46) | (n = 42) | (n = 44) | (95% CI) | P value | (95% CI) | P value |
| Biomarker | 3.60 | 2.63 | 1.00 | 3.58 † | <0.0001 | 2.62 † | <0.0001 |
| response | | | | (2.91-4.31) | | (2.11-3.24) | |
| GLSM* | | | | | | | |

Data are GLSM (95% CI) unless otherwise specified.
GLSM=geometric least squares means
*GLSM of the ratio of max post-op value over baseline.
† GLSM ration represents GLSM in the active treatment group over GLSM in the placebo group.

The unblinded statistician recommended the study be continued without enrichment order to provide additional evidence in support of the secondary outcomes and the study sponsor representative agreed. Therefore, an additional 73 patients (approximately 24 per group) were enrolled without enrichment. The safety population consisted of all randomized patients (n=135), all of whom received study treatment. Of those, 132 (98%) patients had biomarker measurements collected (ITT population), for whom the primary outcome was evaluated (n=46 high-dose, n=42 low-dose, n=44 placebo). From the ITT population, 121 patients had surgery on-time (24 to 48 hours after infusion) and constituted the MITT population, for whom secondary and clinical outcomes were evaluated (n=41 high-dose, n=39 low-dose, n=41 placebo).

At randomization, baseline characteristics were generally similar among intervention groups in the safety population (Supplementary Table S3), ITT population (Table 18), and MITT population (Supplementary Table S4). In the ITT population, the EuroSCORE and AKI risk factors were often numerically lower in the placebo group versus both RBT-1 groups. However, the contrary was observed for the incidence of combined surgery (CABG+Valve). The time between infusion and start of surgery, as well as time on cardiopulmonary bypass, were similar between the 3 treatment groups.

The primary composite outcome in the ITT population was 3.60 in the high-dose RBT-1 group, 2.63 in the low-dose RBT-1 group, and 1.00 in the placebo group (Table 20, FIG. 12). Both high-dose and low-dose RBT-1 were associated with an increased preconditioning biomarker response compared with placebo (high-dose vs. placebo; GLSM ratio, 3.58; 95% CI, 2.91-4.41; p<0.0001; and low-dose vs. placebo; GLSM ratio, 2.62; 95% CI, 2. 11-3·24; p<0.0001).

Secondary outcomes of AKI incidence and sustained reduction in urine output were numerically lower with both doses of RBT-1 compared with placebo but the differences were not significant (Table 20).

TABLE 20

| | High- | Low- | | High-dose v. | Low-dose v. |
|---|---|---|---|---|---|
| | dose | dose | Placebo | placebo, risk ratio | placebo, risk ratio |
| | (n = 41) | (n = 39) | (n = 41) | (95% CI) | (95% CI) |
| Acute kidney injury | 7(17%) | 7(18%) | 8(20%) | 0.88(0.35-2.19) | 0.92(0.37-2.30) |
| Sustained reduction in | 2(5%) | 2(5%) | 4(10%) | 0.5(0.10-2.58) | 0.53(0.10-2.71) |
| urine output | | | | | |
| Tubular injury | 7.9 | 10.8 | 6.1 | 1.29† (0.71-2.35) | 1.78† (0.96-3.30) |
| biomarker | | | | | |
| response, GLSM* | | | | | |
| Biomarker response, | 3.60 | 2.62 | 1.00 | 3.61† (2.91-4.47) | 2.62† (2.11-3.27) |
| GLSM* | | | | | |

Data are n (%) unless otherwise specified. Percentages are rounded.
GLSM=geometic least squares mean.
*GLSM of the ratio of max post-op value over baseline.
†GLSM ratio represents GLSM in the active treatment group over GLSM in the placebo group.

The composite of renal tubular injury biomarkers occurred with similar frequency in the RBT-1 and placebo groups, and these biomarkers did not correlate with the maximum change in serum creatinine (data not shown). The primary composite outcome results in the MITT population were consistent with the results in the ITT population.

RBT-1 was generally well tolerated by patients. The primary drug-related AE was photosensitivity, a known reaction to the SnPP component of RBT-1 (Table 21).

TABLE 21

| | | | | High-dose | Low-dose |
|---|---|---|---|---|---|
| | High- | Low- | | v. placebo, | v. placebo, |
| | dose | dose | Placebo | risk ratio | risk ratio |
| | (n = 41) | (n = 39) | (n = 41) | (95% CI) | (95% CI) |
| Primary drug- | | | | | |
| related adverse | | | | | |
| event | | | | | |
| Photosensitivity | 12 | 6 | 0 | 26.1 | 13.3 |
| Reaction | (26%) | (13%) | (0%) | (14.2 to 41.1) | (3.8 to 27.0) |
| Deaths | | | | | |
| Death | 2 | 1 | 3 | −2.5 | −4.6 |
| | (4%) | (2%) | (7%) | (−15.1 to 8.9) | (−16.5 to 5.8) |
| Adverse Events | | | | | |
| Subjects with | 44 | 40 | 40 | 4.7 | −2.0 |
| at least one | (96%) | (89%) | (91%) | (−6.9 to 17.8) | (−16.2 to 12.0) |
| adverse event | | | | | |

Photosensitivity was dose-dependent, occurring in 12 of 46 patients (26%) treated with high-dose RBT-1 and in 6 of 45 patients (13%) treated with low-dose RBT-1 in the safety population. In general, in intensity. The median time of onset of photosensitivity was 2.0 days in the high-dose RBT-1 group and 2.5 days in the low dose RBT-1 group; median time to resolution was 7.0 days in the high-dose RBT-1 group and 3.5 days in the low-dose RBT-1 group. All photosensitivity reactions resolved within 93 days in the high-dose RBT-1 group and within 28 days in the low-dose RBT-1 group. Three photosensitivity reactions in the high-dose RBT-1 group resulted in delayed surgery. Other AEs of general interest are also provided in Table 21.

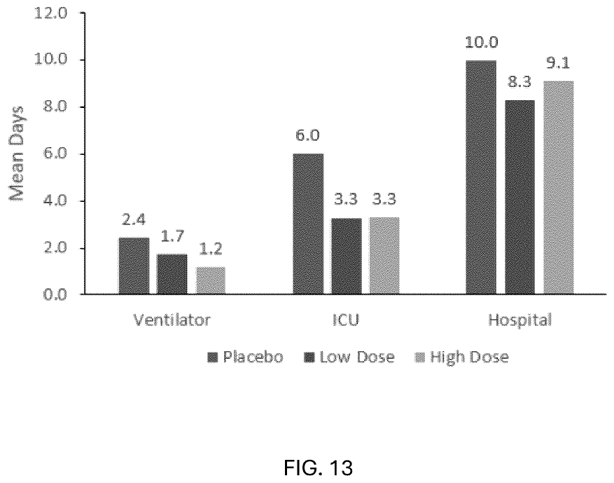

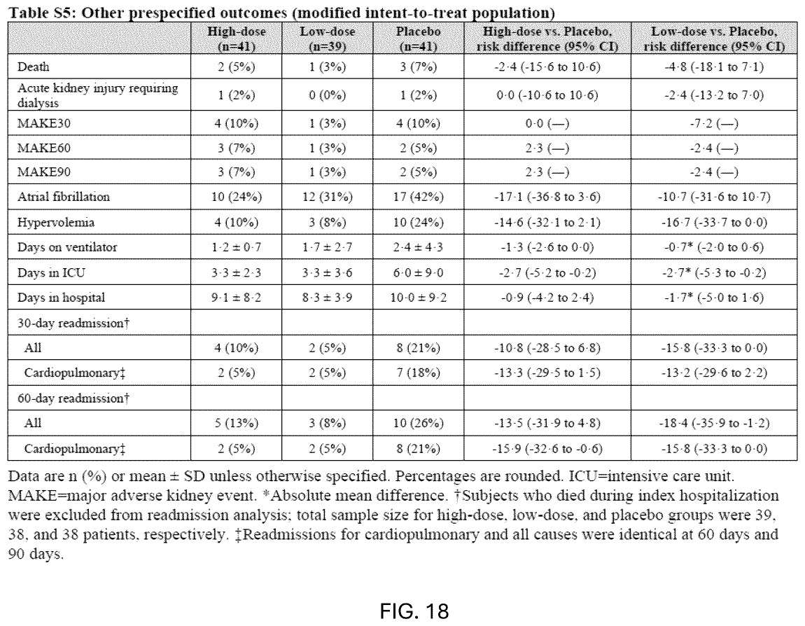

Post-operative complications included 2 (5%) deaths in the high-dose RBT-1 group, 1 (3%) death in the low-dose RBT-1 group, and 3 (7%) deaths in the placebo group FIG. 18 (Table S5). Dialysis for AKI was needed in 1 (2%) patient in the placebo group but none in either RBT-1 group. The number of patients with MAKE was relatively low, and no statistical differences between groups were observed. Post-operative atrial fibrillation and hypervolemia were numerically lower in both RBT-1 groups compared with placebo, but the differences were not significant. The mean time on ventilator was 1.2 days, 1.7 days, and 2.4 days in the high-dose RBT-1, low-dose RBT-1, and placebo groups, respectively. FIG. 13 and FIG. 18 (Table S5). The mean time in ICU was 3.3 days in both RBT-1 groups and 6.0 days in the placebo group. The mean time in hospital was 9. 1 days, 8.3 days, and 10.0 days in the high-dose RBT-1, low-dose RBT-1, and placebo groups, respectively. Finally, 2 (5%) patients each in both RBT-1 groups were readmitted to the hospital at 30-days post-discharge for a cardiopulmonary diagnosis compared with 7 patients (18%) in the placebo group. Through 60 days and 90 days post-discharge, the cardiopulmonary readmission rate remained the same in both RBT-1 groups with 2 (5%) patients each readmitted compared with an increase to 8(21%) patients in the placebo group. All-cause readmissions showed similar results.

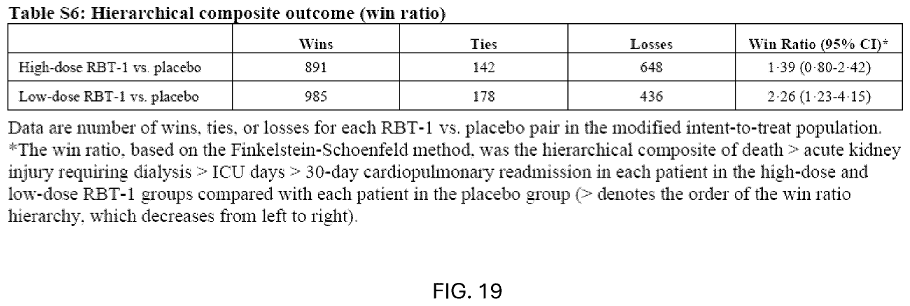

Given the suggested multi-organ benefit of RBT-1, we explored the effects of RBT-1 in a post hoc composite analysis (win ratio) wherein clinical outcomes were assessed in rank order of severity (death>AKI requiring dialysis>ICU days>30-day cardiopulmonary readmission) in the MITT population. The win ratio was 1·39 (95% CI, 0.80-2·42) in patients treated with high dose RBT-1 and 2.26 (95% CI, 1.23-4. 15) with low-dose RBT-1 compared with placebo. FIG. 19 (Table S6).

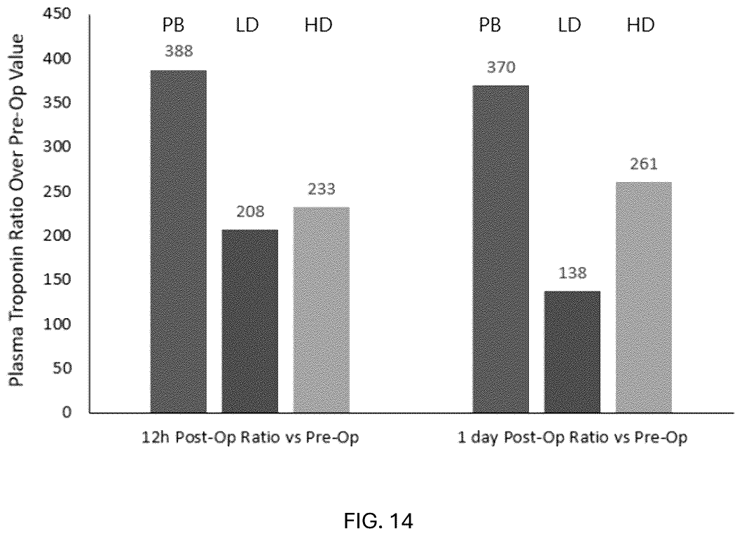

In an analysis of myocardial injury using troponin I, the rise in troponin I after cardiac surgery (GLSM of the ratio between 1-day post-operative values and preoperative baseline values) was numerically lower in the high-dose RBT-1 group compared with placebo but the difference was not significant (GLSM ratio, 0.71; 95% CI, 0.32-1.58). However, the rise in postoperative troponin I was reduced in the low-dose RBT-1 group compared with placebo (GLSM ratio, 0.37; 95% CI, 0-17-0.82) FIG. 14 shows the increase in plasma troponin I from pre-operative baseline to 12 hours (left series) and 1 day (right series) after cardiac surgery. The analysis population, derived from the modified intent-to treat population, excluded patients who had undergone mitral valve repair or replacement, ablation, or septal myectomy due to the expected significant increase in troponin I levels following these major surgeries. The analysis population, derived from the MITT population, excluded patients who had undergone mitral valve repair or replacement, ablation, or septal myectomy due to the expected large increase in troponin I levels following these major surgeries.

### DISCUSSION

This study of RBT-1 met its primary outcome, demonstrating a statistically significant increase in the levels of cytoprotective proteins (plasma HO-1, IL-10, and ferritin), which are surrogate measures for RBT-1-mediated activation of a preconditioning response. The RBT-1 biomarker response was consistent with the Phase 1b study. The overall incidence of AKI was low and differences in AKI-related outcomes were not significant 430 in this population. Despite the relatively small sample size of this study, we observed a reduction in adverse post-operative outcomes, suggesting that RBT-1 may improve recovery after cardiac surgery.

Cardiac surgery, especially with cardiopulmonary bypass, induces systemic inflammation, which can lead to multi-organ dysfunction, impacting clinical outcomes. Importantly, inflammation and oxidative stress exist in a feed-forward loop, magnifying the response of each pathway. The detrimental effect of these cell-damaging mediators can be seen in the phenomenon of “organ crosstalk,” wherein damage in one organ leads to damage in another.

The benefits observed with RBT-1 are likely related to mitigation of these adverse effects by activating anti-inflammatory and antioxidant pathways prior to surgery, thereby resulting in direct and indirect beneficial effects in various organs. For example, the RBT-1-mediated anti inflammatory response may prevent extravasation of fluid into tissues due to capillary leakage, thus reducing the need for fluid (volume) replacement and ultimately fluid overload as observed by the reduction in hypervolemia in RBT-1-treated patients. The broad organ protective benefits of RBT-1 may result in an improvement in clinical outcomes as manifested by reduced time on ventilator, time in ICU, need for vasopressors, new-onset atrial fibrillation, and fluid overload in the short-term and a decrease in cardiopulmonary hospital readmissions in the long-term. To further assess this hypothesis, we explored the effects of RBT-1 in a post hoc analysis using a win ratio based on a composite of clinical outcomes assessed in rank order of severity. This assessment, which consisted of death, AKI requiring dialysis, ICU days, and 30-day cardiopulmonary readmission, suggested clinical improvement in response to RBT-1, which will be confirmed in an upcoming Phase 3 study.

The safety profile of RBT-1 showed that it was well tolerated, with the primary drug related AE being photosensitivity, which was dose-related and time-limited. The SnPP component (a metalloporphyrin) of RBT-1 is likely the cause of photosensitivity as metalloporphyrins are light responsive and may lead to a sunburn in patients exposed to the sun, especially if sun exposure is prolonged or sunscreen is not used. The low-dose of RBT-1 (45 mg SnPP/240 mg FeS) is planned for the definitive Phase 3 trial due to comparable efficacy and fewer photosensitivity reactions compared with high-dose RBT-1.

As a Phase 2 trial, the aim was to investigate whether RBT-1 administered before surgery would elicit a preconditioning response in patients undergoing CABG and/or heart valve surgery and was not powered (i.e., relatively small sample size) to demonstrate statistically significant reductions in clinical outcomes; a larger Phase 3 trial is needed to demonstrate such effects. This study was conducted during the COVID-19 pandemic, which may have impacted the LOS in ICU; however, to minimize variability in standard of care, patients were randomized at the site level. Although composite outcomes showing statistically significant improvement were evaluated post hoc, a consistent trend of improvement with RBT-1 treatment was observed. One (0.7%) patient had incomplete serum creatinine values required for the MAKE outcome at Day 60 and 90 due to COVID-19 that prevented the patient from returning to the hospital for lab collection. However, we imputed laboratory values for this patient. Given the exploratory nature of the trial, the type I error rate was not controlled using bias adjustment methods.

This study of RBT-1 met its primary outcome, demonstrating a statistically significant increase in the levels of cytoprotective proteins (plasma HO-1, IL-10, and ferritin), which are surrogate measures for RBT-1-mediated activation of a preconditioning response. Given the positive trends in clinical outcomes and adequate safety profile, a Phase 3 study of RBT-1 is planned, wherein the primary outcome will be a hierarchical composite of clinical outcomes.

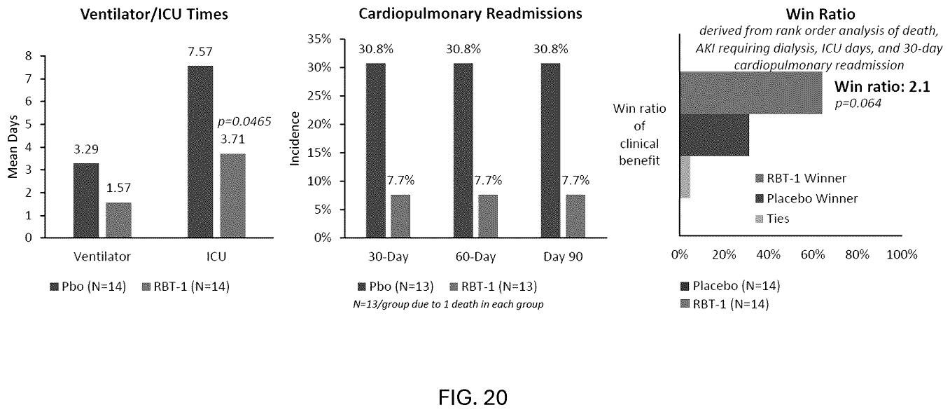

Patients undergoing CABG/cardiac value surgery showed an improvement in clinical outcomes, including (a) ventilator time, (b) ICU time, and (c) cardiopulmonary readmission as shown in FIG. 20. Specifically, the ventilator time was reduced from 3.29 for the placebo group to 1.57 for the RBT-1 treatment group. The ICU time was reduced from 7.57 for the placebo group to 3.71 for the RBT-1 treatment group. The 30-, 60-, and 90- day cardiopulmonary readmissions were reduced from 30.8% for the placebo group to 7.7% for the RBT-1 treatment group. These results are further demonstrated by a win ratio of 2.1 for RBT-1 versus placebo as shown in FIG. 20.

Additional observations from the study include the following:

A significant reduction of 49% in the incidence of post-operative anemia, as reported by the investigators, was observed in response to RBT-1 compared with placebo (p=0.0447).

A 33% reduction in the need for blood transfusion was observed in the RBT-1 group compared with placebo.

Similarly, a 42% reduction in the need for iron supplementation was observed in the RBT-1 group compared with placebo.

When comparing all patients who required both blood transfusion and iron, a statistically significant reduction was observed in the RBT-1 group compared with placebo (RBT-1: 0.0% vs Placebo: 12.2%; p=0.0046).

Importantly, hemoglobin levels at discharge in patients who received RBT-1 were comparable to those who received placebo despite lesser rates of blood transfusion and iron supplementation (RBT-1: 9.8 g/dL vs Placebo: 9.8 g/dL).

### Example 4

A patient who has been found to be photosensitive (skin is susceptible to sunburn) is treated by intravenous infusion of RBT-1 between 24 and 48 hours before a scheduled CABG surgery. The RBT-1 dose is 45 mg stannic protoporfin and 240 mg iron sucrose. After infusion of RBT-1 and before any exposure to sunlight, the patent's exposed skin is treated with SPF 50 sunscreen. The patent undergoes CABG surgery. After surgery, SPF 50 sunscreen is administered prior to any sunlight exposure within six days from the surgery.

### Example 5

A patient who has been found to be photosensitive (skin is susceptible to sunburn) is treated by intravenous infusion of RBT-1 between 24 and 48 hours before a scheduled CABG surgery. The RBT-1 dose is 45 mg stannic protoporfin and 240 mg iron sucrose. Exposure to sunlight is avoided from the time of RBT-1 administration and the surgery. The patent undergoes CABG surgery. After surgery, SPF 50 sunscreen is administered prior to any sunlight exposure within six days from the surgery.

### Example 6

A patient who is susceptible to hospital readmission for cardiopulmonary purposes after CABG surgery is treated by is treated by intravenous infusion of RBT-1 between 24 and 48 hours before a scheduled CABG surgery. The RBT-1 dose is 45 mg stannic protoporfin and 240 mg iron sucrose. The patent undergoes CABG surgery. After surgery, the patient is at least 60% less likely to need readmission to the hospital for cardiopulmonary purposes.

### Example 7

A patient who is susceptible post-operative complications after CABG surgery is treated by is treated by intravenous infusion of RBT-1 between 24 and 48 hours before a scheduled CABG surgery. The RBT-1 dose is 45 mg stannic protoporfin and 240 mg iron sucrose. The patent undergoes CABG surgery. After surgery, the patient is less likely to suffer from the following complications (a) greater than three days in the intensive care unit, (b) greater than 24 hours on a ventilator, (c) readmission for cardiopulmonary surgery, (d) need for a blood transfusion, (e) new onset post-operative atrial fibrillation (POAF) during hospitalization, or a combination of two or more of (a)-(e).

### Example 8

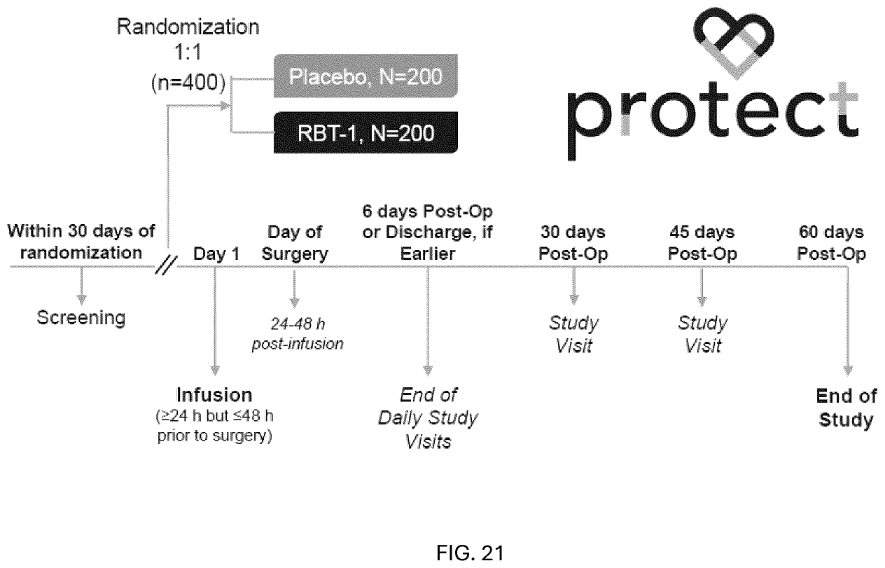

A phase III study is conducted for evaluating reducing the risk of postoperative complications in patients undergoing cardiothoracic surgery. FIG. 21 shows the design of this study. The primary objective of the study will be to evaluate the efficacy of RBT-1 compared with placebo on a hierarchical composite (win ratio) of: death, incidence of acute kidney injury (AKI) requiring dialysis, intensive care unit (ICU) days, and 30-day cardiopulmonary readmission rates. The secondary objective of the study will focus on post-operative complications, ICU days, 30-day cardiopulmonary readmission rates, and safety. The post-operative complications that will be studied include death, AKI requiring dialysis, >3 days in ICU, >24 hours on a ventilator, 30-day cardiopulmonary readmission, need for blood transfusion during index hospitalization, and new-onset post-operative atrial fibrillation (POAF) during index hospitalization.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

What is claimed is:

1. A method of reducing post operative complications of a human patient from injury based on a scheduled or anticipated surgical operation, the method comprising administering to the human patient a composition comprising a therapeutically effective amount of (i) an iron compound; and (ii) stannic protoporfin in a dose of 20-80 mg before the surgical operation, wherein the human patient is susceptible to photodermatoses.

2. The method of claim 1, wherein the stannic protoporfin is administered in a dose of 30-70 mg.

3. The method of any of claim 1 or 2, wherein the stannic protoporfin is administered in a dose of 45 mg.

4. The method of any of claims 1-3, wherein the iron compound is iron sucrose is administered at a dose of 190-290 mg.

5. The method of any of claims 1-4, wherein the iron compound is iron sucrose is administered at a dose of 240 mg.

6. The method of any of claims 1-5, wherein the stannic protoporfin is administered at a dose of 45 mg and iron compound is iron sucrose is administered at a dose of 240 mg.

7. The method of any of claims 1-6, wherein the sunscreen is applied to the patient's skin within six days after the scheduled or anticipated surgical operation.

8. The method of claim 7, wherein sunscreen is applied to the patient's skin before any expected sun exposure within six days after the scheduled or anticipated surgical operation.

9. The method of any of claims 1-8, wherein the human patient avoids sun exposure between the administration of the therapeutically effective composition and the scheduled or anticipated surgical operation, and wherein the human patient applies sunscreen when sunlight exposure is expected for at least 6 days after the scheduled or anticipated surgical operation.

10. The method of any of claims 1-9, wherein the human patient applies sunscreen when sun exposure is expected after the therapeutically effective composition is administered.

11. The method of any of claims 1-10, wherein the sunscreen has a sun protection factor (SPF) of 50+.

12. The method of any of claims 1-11, wherein the iron compound is present in an aqueous pharmaceutical composition comprising: iron sucrose; bicarbonate; and a pharmaceutically acceptable aqueous carrier.

13. The method of any of claims 1-12, wherein the stannic protoporfin is present in an aqueous pharmaceutical composition comprising: a total level of impurities of 1.5% or less, as measured by gas chromatography.

14. The method of any of claims 1-13, wherein preparing the therapeutically effective composition comprises combining in an intravenous bag:
a. an aqueous iron pharmaceutical composition comprising: iron sucrose; bicarbonate; and a pharmaceutically acceptable aqueous carrier; and
b. a stannic protoporfin composition having a total level of impurities of 1.5% or less, as measured by gas chromatography.

15. The method of any of claims 1-14, wherein the therapeutically effective amount of stannic protoporfin is 0.5-0.8 mg/kg.

16. The method of any of claims 1-15, wherein the therapeutically effective amount of stannic protoporfin is 0.6-0.7 mg/kg.

17. The method of any of claims 1-16, wherein the therapeutically effective amount of stannic protoporfin is 0.63 mg/kg.

18. The method of any of claims 1-17, wherein the therapeutically effective amount of iron sucrose is 2-6 times the amount of stannic protoporfin.

19. The method of any of claims 1-18, wherein the therapeutically effective amount of iron sucrose is 3-4 times the amount of stannic protoporfin.

20. The method of any of claims 1-19, wherein the scheduled or anticipated surgical operation is surgery to an organ.

21. The method of claim 20, wherein the organ is a heart.

22. The method of claim 20, wherein the organ is a kidney.

23. The method of claim 20, wherein the organ is a liver.

24. The method of claim 20, wherein the organ is a lung.

25. The method of any of claims 1-24, wherein the iron compound and the stannic protoporfin are administered at least 24 hours before the scheduled or anticipated surgical operation to the organ occurs.

26. The method of any of claims 1-25, wherein the iron compound and the stannic protoporfin are administered no more than 48 hrs before the scheduled or anticipated surgical operation to the organ occurs.

27. The method of any of claims 1-26, wherein scheduled or anticipated surgical operation is a surgery comprising coronary artery bypass graft (CABG), cardiac valve, or combined CABG/valve surgery on cardiopulmonary bypass (CPB).

28. The method of any of claims 1-27, wherein the scheduled or anticipated surgical operation is organ transplant surgery.

29. The method of any of claims 1-28, wherein the method reduces or eliminates the need for a blood product to be administered after the scheduled or anticipated surgical operation.

30. The method of any of claims 1-29, wherein the blood products comprises whole blood from a donor.

31. The method of any of claims 1-30, wherein the method reduces the need for hospital readmission for cardiopulmonary purposes.

32. The method of any of claims 1-31, wherein the method reduces a post operative complication of the scheduled or anticipated surgical operation.

33. The method of claim 32, wherein the post operative complications comprises greater than three days in the intensive care unit, greater than 24 hours on a ventilator, readmission for cardiopulmonary surgery, need for a blood transfusion, new onset post-operative atrial fibrillation (POAF) during hospitalization, or a combination of two or more thereof.

34. A method of reducing post operative complications of a human patient from injury based on a scheduled or anticipated insult to an organ of the human patient, the method comprising:
(a) administering to the patient a therapeutically effective composition comprising (i) an iron compound; and (ii) stannic protoporfin, and
(b) applying sunscreen to the human patient's skin around the time of administering to the human patient the therapeutically effective composition or within six days after the scheduled or anticipated surgical operation,
wherein the therapeutically effective composition is administered before the scheduled or anticipated insult to the organ occurs.

35. The method of claim 34, wherein step (a) comprises administering stannic protoporfin in a dose of 20-80 mg.

36. The method of any of claims 34-35, wherein the stannic protoporfin is administered in a dose of 30-70 mg.

37. The method of any of claims 34-36, wherein the stannic protoporfin is administered in a dose of 45 mg.

38. The method of any of claims 34-37, wherein the iron sucrose is administered at a dose of 190-290 mg.

39. The method of any of claims 34-38, wherein the iron sucrose is administered at a dose of 240 mg.

40. The method of any of claims 34-39, wherein the stannic protoporfin is administered at a dose of 45 mg and iron sucrose is administered at a dose of 240 mg.

41. The method of any of claims 34-40, further comprising applying sunscreen to the patient's skin, wherein the sunscreen protects the patient while the patient is exposed to the therapeutically effective composition.

42. The method of any of claims 34-41, wherein the step of administering to the patient the therapeutically effective composition is performed before applying sunscreen to the patient's skin.

43. The method of any of claims 34-42, wherein the patient avoids sun exposure between the administration of the therapeutically effective composition and the insult, and wherein the patient applies sunscreen for at least 6 days post-surgery.

44. The method of any of claims 34-43, wherein the patient applies sunscreen when sun exposure is expected after the therapeutically effective composition is administered.

45. The method of any of claims 34-44, wherein the sunscreen has a sun protection factor (SPF) of 50+.

46. The method of any of claims 34-45, wherein the iron compound is present in an aqueous pharmaceutical composition comprising: iron sucrose; bicarbonate; and a pharmaceutically acceptable aqueous carrier, wherein the iron sucrose is present in pharmaceutically effective amount, the iron being present in both iron (II) and iron (III) form, the pharmaceutical composition has a pH greater than 9, a concentration of iron (II) of 0.05% w/v to 0.41% w/v, and the iron sucrose has a MW according to GPC of between 33,000 and 38,000 Daltons.

47. The method of any of claims 34-46, wherein the stannic protoporfin is present in an aqueous pharmaceutical composition comprising: a total level of impurities of 1.5% or less, as measured by gas chromatography.

48. The method of any of claims 34-47, wherein preparing the therapeutically effective composition comprises combining in an intravenous bag:
a. an aqueous iron pharmaceutical composition comprising: iron sucrose; bicarbonate; and a pharmaceutically acceptable aqueous carrier, wherein the iron sucrose is present in pharmaceutically effective amount for providing a protective effect to a patient's kidney, the iron being present in both iron (II) and iron (III) form, the pharmaceutical composition has a pH greater than 9, a concentration of iron (II) of 0.05% w/v to 0.41% w/v, and the iron sucrose has a MW according to GPC of between 33,000 and 38,000 Daltons; and
b. a stannic protoporfin composition having a total level of impurities of 1.5% or less, as measured by gas chromatography.

49. The method of any of claims 34-48, wherein the therapeutically effective amount of stannic protoporfin is 0.5-0.8 mg/kg.

50. The method of any of claims 34-49, wherein the therapeutically effective amount of stannic protoporfin is 0.6-0.7 mg/kg.

51. The method of any of claims 34-50, wherein the therapeutically effective amount of stannic protoporfin is 0.63 mg/kg.

52. The method of any of claims 34-51, wherein the therapeutically effective amount of iron sucrose is 2-6 times the amount of stannic protoporfin.

53. The method of any of claims 34-52, wherein the therapeutically effective amount of iron sucrose is 3-4 times the amount of stannic protoporfin.

54. The method of any of claims 34-53, wherein the insult is surgery.

55. The method of any of claims 34-54, wherein the organ is a heart.

56. The method of any of claims 34-55, wherein the organ is a kidney.

57. The method of any of claims 34-56, wherein the organ is a liver.

58. The method of any of claims 34-57, wherein the organ is a lung.

59. The method of any of claims 34-58, wherein the iron sucrose and the stannic protoporfin are administered at least 24 hours before the scheduled or anticipated insult to the organ occurs.

60. The method of any of claims 34-59, wherein the iron sucrose and the stannic protoporfin are administered no more than 48 hrs before the scheduled or anticipated insult to the organ occurs.

61. The method of any of claims 34-60, wherein the insult is surgery including coronary artery bypass graft (CABG), cardiac valve, or combined CABG/valve surgery on cardiopulmonary bypass (CPB).

62. The method of any of claims 34-61, wherein the insult is organ transplant surgery.

63. The method of any of claims 34-62, wherein the method reduces or eliminates the need for blood products to be administered after surgery.

64. The method of any of claims 34-63, wherein the blood products comprise whole blood from donors.

65. The method of any of claims 34-64, wherein the method reduces the need for hospital readmission for cardiopulmonary purposes.

66. The method of any of claims 34-65, wherein the method reduces post operative complications of the surgery.

67. The method of claim 66, wherein the post operative complications include: greater than three days in the intensive care unit, greater than 24 hours on a ventilator, readmission for cardiopulmonary surgery, need for a blood transfusion, and/or new onset post-operative atrial fibrillation (POAF) during hospitalization.

68. A method of reducing hospital readmission for cardiopulmonary purposes of a human patient after a surgical operation by at least 60% comprising administering to the patient a therapeutically effective composition comprising an amount of (i) an iron compound; and (ii) stannic protoporfin before the surgical operation.

69. The method of claim 66, wherein the hospital readmission for cardiopulmonary purposes is reduced by at least 70%.

70. The method of claim 66, wherein the hospital readmission for cardiopulmonary purposes is reduced by at least about 72%.

71. A method of reducing post operative complications of a human patient from injury based on a scheduled or anticipated surgical operation comprising administering to the patient a therapeutically effective composition comprising an amount of (i) an iron compound; and (ii) stannic protoporfin before the surgical operation, wherein the post operative complications include: (a) greater than three days in the intensive care unit, (b) greater than 24 hours on a ventilator, (c) readmission for cardiopulmonary surgery, (d) need for a blood transfusion, (e) new onset post-operative atrial fibrillation (POAF) during hospitalization, or a combination of two or more of (a)-(e).

72. The method of any of claims 68-71, wherein the stannic protoporfin is administered in a dose of 30-70 mg.

73. The method of any of claims 68-71, wherein the stannic protoporfin is administered in a dose of 45 mg.

74. The method any of claims 68-73, wherein the iron compound is iron sucrose is administered at a dose of 190-290 mg.

75. The method of any of claims 68-74, wherein the iron compound is iron sucrose is administered at a dose of 240 mg.

76. The method of any of claims 68-75, wherein the stannic protoporfin is administered at a dose of 45 mg and iron compound is iron sucrose is administered at a dose of 240 mg.

77. The method of any of claims 68-76, wherein the iron compound is present in an aqueous pharmaceutical composition comprising: iron sucrose; bicarbonate; and a pharmaceutically acceptable aqueous carrier.

78. The method of any of claims 68-77, wherein the stannic protoporfin is present in an aqueous pharmaceutical composition comprising: a total level of impurities of 1.5% or less, as measured by gas chromatography.

79. The method of any of claims 68-78, wherein preparing the therapeutically effective composition comprises combining in an intravenous bag:
a. an aqueous iron pharmaceutical composition comprising: iron sucrose; bicarbonate; and a pharmaceutically acceptable aqueous carrier; and
b. a stannic protoporfin composition having a total level of impurities of 1.5% or less, as measured by gas chromatography.

80. The method of any of claims 68-79, wherein the therapeutically effective amount of stannic protoporfin is 0.5-0.8 mg/kg.

81. The method of any of claims 68-80, wherein the therapeutically effective amount of stannic protoporfin is 0.6-0.7 mg/kg.

82. The method of any of claims 68-81, wherein the therapeutically effective amount of stannic protoporfin is 0.63 mg/kg.

83. The method of any of claims 68-82, wherein the therapeutically effective amount of iron sucrose is 2-6 times the amount of stannic protoporfin.

84. The method of any of claims 68-83, wherein the therapeutically effective amount of iron sucrose is 3-4 times the amount of stannic protoporfin.

85. The method of any of claims 68-84, wherein the scheduled or anticipated surgical operation is surgery to an organ.

86. The method of claim 85, wherein the organ is a heart.

87. The method of claim 85, wherein the organ is a kidney.

88. The method of claim 85, wherein the organ is a liver.

89. The method of claim 85, wherein the organ is a lung.

90. The method of any of claims 68-89, wherein the iron compound and the stannic protoporfin are administered at least 24 hours before the scheduled or anticipated surgical operation to the organ occurs.

91. The method of any of claims 68-90, wherein the iron compound and the stannic protoporfin are administered no more than 48 hrs before the scheduled or anticipated surgical operation to the organ occurs.

92. The method of any of claims 68-91, wherein scheduled or anticipated surgical operation is a surgery comprising coronary artery bypass graft (CABG), cardiac valve, or combined CABG/valve surgery on cardiopulmonary bypass (CPB).

93. The method of any of claims 68-92, wherein the scheduled or anticipated surgical operation is organ transplant surgery.

94. The method of any of claims 68-93, wherein the method reduces or eliminates the need for a blood product to be administered after the scheduled or anticipated surgical operation.

95. The method of any of claims 68-94, wherein the blood products comprises whole blood from a donor.

96. The method of any of claims 68-95, wherein the method reduces the need for hospital readmission for cardiopulmonary purposes.

97. The method of any of claims 68-96, wherein the method reduces a post operative complication of the scheduled or anticipated surgical operation.

98. The method of claim 97, wherein the post operative complications comprises greater than three days in the intensive care unit, greater than 24 hours on a ventilator, readmission for cardiopulmonary surgery, need for a blood transfusion, new onset post-operative atrial fibrillation (POAF) during hospitalization, or a combination of two or more thereof.
